(19) United States
(12) Patent Application Publication
(10) Pub. No.: US 2021/0238672 A1
(43) Pub. Date: Aug. 5, 2021

(54) SYSTEMS AND METHODS FOR DETECTION OF BIOMOLECULES

(71) Applicant: Quantum Biosystems Inc., Osaka (JP)

(72) Inventors: Eric S. NORDMAN, Palo Alto, CA (US); Valentin DIMITROV, Palo Alto, CA (US); Gaurav GOYAL, Gothenburg (SE); Jessica KILLIAN, Redwood City, CA (US); Kazusuke MIHARA, Kyoto (JP)

(21) Appl. No.: 17/238,090

(22) Filed: Apr. 22, 2021

Related U.S. Application Data
(63) Continuation of application No. PCT/JP2020/047339, filed on Dec. 18, 2020.
(60) Provisional application No. 62/950,049, filed on Dec. 18, 2019.

(51) Int. Cl. C12Q 1/6869; C12Q 1/00; C12Q 1/48

(52) U.S. Cl. CPC C12Q 1/6869; C12Q 1/48; C12Q 1/005

(57) ABSTRACT

The present disclosure provides methods and systems for detecting or analyzing a biomolecule. The methods and systems may comprise the use of a plurality of electrodes and a solution which may comprise a variety of reagents. In an example, the solution may comprise one or more redox-active molecules or compounds which may undergo or facilitate a redox cycling process. The redox cycling process may generate an electric signal that may be measured by one or more of the plurality of electrodes. An introduction of a nucleotide having a redox cycling current modifying particle coupled thereto to the solution may result in a change in the electric signal. The change in the electric signal may be used to identify the biomolecule or a portion thereof.

### CROSS-REFERENCE

This application is a continuation of International Application No. PCT/JP2020/047339, filed Dec. 18, 2020, which claims the benefit of U.S. Provisional Patent Application Ser. No. 62/950,049, filed Dec. 18, 2019, which is entirely incorporated herein by reference.

### BACKGROUND

The ability to detect individual molecular binding events presents numerous potential applications in science and technology which may not be accessible via traditional bulk biochemical assays. For example, single-molecule detection can be beneficial in nucleic acid sequencing. In the absence of single-molecule detection, sequencing methods may be required to employ the technique of clonal amplification, a costly process which may introduce amplification errors into the sequence and may not be capable of interrogating heterogeneities within a sample. Additionally, clonal amplification can eliminate epigenetic modifications in DNA and/or RNA, which modifications are of wide and intense interest across academic, industrial, and clinical settings. Single-molecule sequencing by synthesis may avoid the expenses and issues inherent to clonal amplification and provide more accurate sequencing results. By avoiding dephasing problems, single-molecule sequencing may enable longer read lengths. Epigenetic modifications and heterogeneities within a sample may be preserved.

Currently, single-molecule sequencing systems often use optical detection of fluorescent moieties, which can require expensive laser and camera subsystems, and can be severely limited in bandwidth due to the camera speeds and in throughput due to the diffraction limit of light requiring low sensor density. The higher sample preparation cost and additional hardware cost can make DNA or RNA sequencing more expensive. Hence, it can be advantageous to develop a single-molecule detection system that can offer the potential for high sampling rates and sensor density, as well as scalability and throughput to reduce the cost.

### SUMMARY

The present disclosure presents a method for detecting individual molecular binding events which may, in one application, be used to analyze the sequence and epigenetic content of a nucleic acid molecule. The method may comprise the use of a plurality of electrodes (e.g., a plurality of nanoelectrodes) and a solution comprising a molecular recognition entity. The molecular recognition entity may comprise e.g., a polymerizing enzyme coupled to a nucleic acid molecule, or any other type of enzyme, an antigen, or another type of molecular binding moiety or scaffold, In some cases, the solution may further comprise a molecular binding target such as a nucleotide, substrate, antibody, or other molecules which may bind to or associate with the molecular recognition entity. The solution may further comprise a redox-active moiety. The redox-active moiety may be a molecule or compound which is capable of redox cycling. The redox-active moiety may be a reversible redox moiety. The reversible redox moiety may facilitate redox cycling to generate an electrical current. The redox cycling may occur when one or more reversible redox moieties diffuse in a region and undergo redox cycling in the plurality of electrodes.

The method may further comprise bringing the particle-coupled binding target in contact with the molecular recognition entity under conditions such that the target can bind to the molecular recognition entity. Such a binding event can effect or cause a change in the electrical current detected by the plurality of electrodes. The presence, lack of presence, magnitude, temporal dynamics, or other aspects of the change in the electrical current detected can be used to identify one or more molecular binding events, which in turn, can be used to identify at least a portion (e.g., a single base) of a sequence the target.

The plurality of electrodes may be closely spaced nanoelectrodes. The plurality of the electrodes may be within or adjacent to a gap, pore, well, or nanocavity forming a sensing area. A device may contain any number of such sensing areas from one to hundreds of millions, to more than one billion. The plurality of electrodes can comprise additional nanoelectrodes external to the sensing area. The molecular recognition entity can be immobilized within the sensing area. The particle can comprise oxides such as silicon dioxide, a polymeric material, metallic material, ceramic material, or composites and combination thereof. The particle may be functionalized. The particle may be porous. The polymeric material can be selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), and derivatives thereof.

In some cases, the method of the present disclosure can be used to investigate the sequence and epigenetic modifications of nucleic acids. As an example, the molecular recognition entity may be a polymerizing enzyme bound to a primed nucleic acid template. The polymerizing enzyme may be immobilized within the sensing area. The nucleic acid may be immobilized within the sensing area. The nucleic acid template may be RNA or DNA, and the priming strand may be RNA or DNA independent of the identity of the template strand. The polymerizing enzyme may be a deoxyribonucleic acid (DNA) polymerase (DNAP), a reverse transcriptase (RT), an RNA-dependent RNA polymerase or an RNA polymerase (RNAP). The method can further comprise introducing a particle (e.g. a nanoparticle) having one or more nucleotides coupled thereto under conditions such that at least one nucleotide can be incorporated into the nucleic acid primer strand by the polymerase. The one or more nucleotides can be coupled to the particle by a tether. The tether can be coupled to the nucleotide through a phosphate group of the nucleotide such that the particle can be decoupled from the nucleotide when the nucleotide is incorporated into the nucleic acid strand. The tether can comprise a polymeric material such as an alkane polymer. The polymeric material can be selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), and a derivative thereof.

The method can further comprise bringing the polymerizing enzyme bound to a primed nucleic acid template in contact with an additional nucleotide having an additional particle coupled thereto, under conditions such that the additional nucleotide can be incorporated into the nucleic acid strand by the polymerizing enzyme, and the additional nucleotide can be coupled to the additional particle by an additional tether. The tether and the additional tether can have different lengths. The particle can be functionalized with a plurality of polymers on a surface of the particle at a density that can reduce non-specific binding to other surfaces. The plurality of polymers can comprise an alkane polymer. The plurality of polymers can comprise a polymer that can be selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), and any combination thereof.

The method can further comprise bringing the polymerizing enzyme bound to a primed nucleic acid template in contact with an additional nucleotide having an additional particle coupled thereto, under conditions such that the additional nucleotide can be incorporated into the nucleic acid strand by the polymerizing enzyme. One or both of the particles and the additional particle can comprise a plurality of polymers as discussed above. The particle may comprise the plurality of polymers at a different density than the additional particle. The particle can have a charge or molecular functional group that electrostatically attracts the reversible redox moiety towards the particle.

The method can further comprise using a lack of a change in the detected electrical current to identify an abasic site on the nucleic acid strand.

The polymerizing enzyme bound to a primed nucleic acid template can be contacted with a first buffer comprising a first cation or a mixture of cations. The first buffer may facilitate a transient binding of the nucleotide at the polymerizing enzyme's active site. During the transient binding, binding kinetics may be measured. The polymerizing enzyme can be subsequently contacted with a second buffer comprising a second cation or a mixture of cations to facilitate incorporation of the nucleotide into the nucleic acid template strand. The first cation or the mixture thereof can comprise Ca²⁺, Zn²⁺, Fe²⁺, Be²⁺, Ba²⁺, Cd²⁺, Co²⁺, Cr²⁺, Ni²⁺, Cu²⁺, Pb²⁺, Sr²⁺, or any combination thereof. The second cation or the mixture thereof can comprise Mg²⁺, Mn²⁺, Zn²⁺, Fe²⁺, Be²⁺, Ba²⁺, Cd²⁺, Co²⁺, Cr²⁺, Ni²⁺, Cu²⁺, Pb²⁺, Sr²⁺, or any combination thereof. The first cation can be Ca²⁺ or a mixture containing Ca²⁺ and the second cation can be Mg²⁺ or a mixture containing Mg²⁺. The change in the electrical current can be detected during the transient binding of the nucleotide-particle conjugate to the polymerizing enzyme. In some embodiments, the first cation and the second cation may be the same or different from each other.

An aspect of the present disclosure provides a method for analyzing a nucleic acid molecule, comprising: (a) providing said nucleic acid molecule coupled to a polymerizing enzyme, wherein said polymerizing enzyme is immobilized adjacent to an electrode of an electrode pair; (b) bringing a reversible redox moiety in contact with said electrode pair, wherein said reversible redox moiety facilitates redox cycling to generate an electrical current between said electrode pair; (c) allowing an interrogating nucleotide, coupled to a particle (e.g., a redox cycling current modifying particle), to bind to a nucleic acid strand complementary to said nucleic acid molecule with the aid of said polymerizing enzyme, said redox cycling current modifying particle effecting a change in said electric current between said electrode pair, when said interrogating nucleotide is complementary to said nucleic acid molecule; and (d) using said change in said electrical current to identify said interrogating nucleotide, thereby identifying at least a portion of a sequence of said nucleic acid molecule.

In some embodiments, said electrode pair is part of a plurality of electrode pairs, and wherein at least two electrode pairs of said plurality of electrode pairs comprise a common electrode. In some embodiments, said electrode pair comprises a nanogap comprising a size between about 100 nanometers (nm) and 200 nm. In some embodiments, said electrode pair is disposed within a cavity, wherein a bottom surface of said cavity comprises a first electrode of said electrode pair, and

wherein a rim of said cavity comprises a second electrode of said electrode pair. In some embodiments, a width of said cavity is greater than or equal to a height of said cavity.

In some embodiments, said electrical current between said electrode pair is between about 50 picoamperes (pA) and 300 pA. In some embodiments, said electrical current comprises a noise-level of no more than 2 pA. In some embodiments, said redox cycling current modifying particle comprises a dimension that is at least about 70% of a distance between electrodes of said electrode pair.

In some embodiments, said polymerizing enzyme comprises a deoxyribonucleic acid (DNA) polymerase, a reverse transcriptase, an RNA-dependent RNA polymerase, an RNA polymerase, or any combination thereof. In some embodiments, said interrogating nucleotide is coupled to said particle by a terminal phosphate of said interrogating nucleotide.

In some embodiments, said change in said electric current comprises a decrease in intensity of at least 10% of said electric current. In some embodiments, said change in said electric current comprises a signal-to-noise ratio of at least about 3.

In some embodiments, said particle is coupled to at least two types of nucleotides. In some embodiments, the method further comprises contacting said polymerizing enzyme with a divalent cation to facilitate decoupling of said nucleotide from said particle, thereby releasing said particle.

Another aspect of the present disclosure provides a method for analyzing a nucleic acid molecule, comprising: (a) introducing a solution comprising a reversible redox moiety to an electrode pair having immobilized adjacent thereto a polymerizing enzyme coupled to said nucleic acid molecule, wherein said reversible redox moiety facilitates redox cycling to generate an electrical current between said electrode pair; (b) bringing said nucleic acid molecule in contact with a nucleotide having a particle coupled thereto, under conditions such that said nucleotide couples to a nucleic acid strand complementary to said nucleic acid molecule with aid of said polymerizing enzyme, wherein said particle affects a change in said electrical current; and (c) using said change in said electrical current to identify said nucleotide coupled to said nucleic acid strand, thereby identifying at least a portion of a sequence of said nucleic acid molecule.

In some embodiments, at least a portion of said solution is within a well, and wherein at least one electrode of said electrode pair is disposed in said well. In some embodiments, said well is disposed in a substrate comprising a plurality of wells, wherein said plurality of wells comprises a plurality of electrode pairs comprising said electrode pair.

In some embodiments, said nucleic acid molecule is immobilized to an internal surface of said well or to a support in said well. In some embodiments, said nucleic acid molecule is immobilized to a support of said well. In some embodiments, said polymerizing enzyme is immobilized in the vicinity of or in said well. In some embodiments, said polymerizing enzyme comprises a deoxyribonucleic acid (DNA) polymerase, a reverse transcriptase, an RNA-dependent RNA polymerase, an RNA polymerase, or any combination thereof.

In some embodiments, said particle comprises an oxide. In some embodiments, said particle comprises a polymeric material. In some embodiments, said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), and a derivative thereof. In some embodiments, said particle comprises a metallic material.

In some embodiments, the method further comprises bringing said nucleic acid molecule into contact with an additional nucleotide having an additional particle coupled thereto, under conditions such that said additional nucleotide binds to said nucleic acid strand. In some embodiments, said nucleotide and said additional nucleotide are of different base types, and wherein said particle and said additional particle have substantially identical sizes. In some embodiments, said nucleotide and said additional nucleotide are of different base types, and wherein said particle and said additional particle have different sizes. In some embodiments, said nucleotide and said additional nucleotide are of different base types, and wherein said particle and said additional particle have different surface areas.

In some embodiments, said nucleotide is coupled to said particle by a tether. In some embodiments, said tether is coupled to said nucleotide by a terminal phosphate of a triphosphate group of said nucleotide such that said particle is decoupled from said nucleotide when said nucleotide is incorporated into said nucleic acid strand. In some embodiments, said tether comprises a polymeric material. In some embodiments, said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof. In some embodiments, the method further comprises bringing said nucleic acid molecule into contact with an additional nucleotide having an additional particle coupled thereto, under conditions such that said additional nucleotide binds to said nucleic acid strand, wherein said additional nucleotide is coupled to said additional particle by an additional tether. In some embodiments, said tether and said additional tether have different lengths.

In some embodiments, said particle comprises a plurality of polymers on a surface of said particle at a density that reduces non-specific binding to said surface. In some embodiments, said plurality of polymers comprises a polymer selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof.

In some embodiments, the method further comprises bringing said nucleic acid molecule into contact with an additional nucleotide having an additional particle coupled thereto, under conditions such that said additional nucleotide binds to said nucleic acid strand, wherein said particle and said additional particle comprise said plurality of polymers on surfaces at different densities. In some embodiments, said particle has a charge that electrostatically attracts said reversible redox moiety towards said particle. In some embodiments, the method further comprises, subsequent to (c), using a lack of a change in said electrical current detected in (b) to identify an abasic site on said nucleic acid strand.

In some embodiments, said polymerizing enzyme is contacted with a first buffer comprising a first cation to facilitate a transient binding of said nucleotide with said nucleic acid molecule; and wherein said polymerizing enzyme is subsequently contacted with a second buffer comprising a second cation to facilitate incorporation of said nucleotide into said nucleic acid strand. In some embodiments, said first cation is selected from the group consisting of Ca²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. In some embodiments, said second cation is selected from the group consisting of Mg²⁺, Mn²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. In some embodiments, said first cation is Ca²⁺ and said second cation is Mg²⁺.

In some embodiments, said change in said electrical current is detected during said transient binding of said nucleotide with said nucleic acid molecule. In some embodiments, said change is an increase or a decrease in said electrical current. In some embodiments, said electrode pair comprises a nanoelectrode.

Another aspect of the present disclosure provides a system for analyzing a nucleic acid molecule, comprising: an electrode pair configured to receive and immobilize adjacent a polymerizing enzyme coupled to said nucleic acid molecule, said electrode pair comprising electrodes separated by a gap, which gap is configured to receive at least part of a solution comprising a reversible redox moiety configured to facilitate redox cycling to generate an electrical current between said nanoelectrodes; a controller operatively coupled to said electrode pair, which said controller is configured to (i) detect a change in said electrical current upon a nucleotide having a particle coupled thereto coming into contact with said nucleic acid molecule, such that said nucleotide binds to a nucleic acid strand complementary to said nucleic acid molecule with the aid of said polymerizing enzyme, wherein said particle effects said change in said electrical current, and (ii) use said change in said electrical current detected in (i) to identify said nucleotide, thereby identifying at least a portion of a sequence of said nucleic acid molecule.

In some embodiments, the system further comprises a well configured to contain at least a part of said solution, wherein said well comprises an electrode of said plurality of electrodes. In some embodiments, said controller is configured to select which electrode pairs are addressed. In some embodiments, the system further comprises a fluid flow unit configured to dispense said nucleotide having said particle coupled thereto.

In some embodiments, said nucleic acid molecule is immobilized to a surface internal to said well or to a support in said well. In some embodiments, said nucleic acid molecule is immobilized to said well. In some embodiments, said polymerizing enzyme is immobilized in the vicinity of or in said well. In some embodiments, said polymerizing enzyme comprises a deoxyribonucleic acid (DNA) polymerase, a reverse transcriptase, an RNA-dependent RNA polymerase, an RNA polymerase, or any combination thereof. In some embodiments, said particle is a solid particle which comprises silicon dioxide.

In some embodiments, said particle comprises a polymeric material. In some embodiments, said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), and a derivative thereof. In some embodiments, said particle comprises a metallic material.

In some embodiments, said well is configured to contain an additional nucleotide having an additional particle coupled thereto, under conditions such that said additional nucleotide is incorporated into said nucleic acid strand. In some embodiments, said nucleotide and said additional nucleotide are of different base types, and wherein said particle and said additional particle have substantially identical sizes. In some embodiments, said nucleotide and said additional nucleotide are of different base types, and wherein said particle and said additional particle have different sizes. In some embodiments, said nucleotide and said additional nucleotide are of different base types, and wherein said particle and said additional particle have different surface areas.

In some embodiments, said nucleotide is coupled to said particle by a tether. In some embodiments, said tether is coupled to said nucleotide by a phosphate group of said nucleotide such that said particle is decoupled from said nucleotide when said nucleotide is incorporated into said nucleic acid strand. In some embodiments, said tether comprises a polymeric material. In some embodiments, said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof.

In some embodiments, said well is configured to contain an additional nucleotide having an additional particle coupled thereto, under conditions such that said additional nucleotide binds to said nucleic acid strand, wherein said additional nucleotide is coupled to said additional particle by an additional tether. In some embodiments, said tether and said additional tether have different lengths.

In some embodiments, said particle comprises a plurality of polymers on a surface of said particle at a density configured to reduce non-specific binding to said surface. In some embodiments, said plurality of polymers comprises a polymer selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof. In some embodiments, said well is configured to contain an additional nucleotide having an additional particle coupled thereto, under conditions such that said additional nucleotide binds to said nucleic acid strand, wherein said particle and said additional particle comprise said plurality of polymers on surfaces at different densities. In some embodiments, said particle has a charge that electrostatically attracts said reversible redox moiety.

In some embodiments, said well is configured to contain (iii) a first buffer comprising a first cation in contact with said polymerizing enzyme to facilitate said polymerizing enzyme to transiently bind said nucleotide to said nucleic acid molecule; and (iv) a second buffer comprising a second cation in contact with said polymerizing enzyme to facilitate said polymerizing enzyme to incorporate said nucleotide into said nucleic acid molecule. In some embodiments, said first cation is selected from the group consisting of Ca²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. In some embodiments, said second cation is selected from the group consisting of Mg²⁺, Mn²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺.

In some embodiments, said change in said electrical current is detected during a transient binding of said nucleotide with said nucleic acid molecule. In some embodiments, said electrode pair comprises a nanoelectrode.

Another aspect of the present disclosure provides a method for analyzing a nucleic acid molecule, comprising: (a) providing a plurality of electrodes and a solution comprising said nucleic acid molecule having a polymerizing enzyme coupled thereto, wherein said plurality of electrodes detects an electrical current through said solution; (b) bringing said nucleic acid molecule into contact with a nucleotide having a particle coupled thereto, under conditions such that said polymerizing enzyme facilitates a transient binding of said nucleotide to a nucleic acid strand complementary to said nucleic acid molecule, wherein said transient binding of said nucleotide having said particle coupled thereto effects a change in said electrical current detected by said plurality of electrodes; (c) subsequent to said transient binding, incorporating said nucleotide into said nucleic acid strand; and (d) using said change in said electrical current detected by said plurality of electrodes to identify said nucleotide transiently bound to said nucleic acid strand, thereby identifying at least a portion of a sequence of said nucleic acid molecule.

In some embodiments, said polymerizing enzyme is contacted with a first buffer comprising a first cation to facilitate said transient binding of said nucleotide to said nucleic acid molecule; and wherein said polymerizing enzyme is subsequently contacted with a second buffer comprising a second cation to facilitate incorporation of said nucleotide into said nucleic acid strand. In some embodiments, said first cation is selected from the group consisting of Ca²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. In some embodiments, said second cation is selected from the group consisting of Mg²⁺, Mn²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. In some embodiments, said first cation is Ca²⁺ and said second cation is Mg²⁺.

In some embodiments, said polymerizing enzyme is a deoxyribonucleic acid (DNA) polymerase, a reverse transcriptase, an RNA-dependent RNA polymerase, or an RNA polymerase. In some embodiments, said DNA polymerase is selected from the group consisting of Φ15, Φ29, BS32, B103, Cp-1, Cp-5, Cp-7, GA.-1, G1, L17, M2Y, Nf, PRD1, PZE, PR4, PR5, PR722, PZA, SF5, T4, T7 DNA polymerases, or a functional variant thereof. In some embodiments, said polymerizing enzyme is a modified recombinant polymerase. In some embodiments, said polymerizing enzyme is modified such that it lacks 5′ to 3′ and/or 3′ to 5′ exonuclease activity.

In some cases, the method further comprises determining a binding kinetic (e.g., a binding rate or rate constant or a dissociation rate or rate constant) of said nucleotide to said nucleic acid molecule from said change in said electrical current, and identifying said nucleotide based on said binding kinetic. In some cases, said plurality of electrodes comprises a plurality of nanoelectrodes. In some cases, said solution is contained in a well, and wherein said well comprises a nanoelectrode of said plurality of nanoelectrodes.

Various aspects of the present disclosure provide a system for analyzing a nucleic acid molecule, comprising: a plurality of electrodes, wherein at least two of said plurality of electrodes are separated from each other by a gap, which gap is configured to receive at least part of a solution comprising said nucleic acid molecule having a polymerizing enzyme coupled thereto; a controller operatively coupled to said plurality of electrodes, which said controller is configured to (i) use said plurality of electrodes to detect a change in said electrical current upon a nucleotide having a particle coupled thereto coming into contact with said nucleic acid molecule, under conditions such that said polymerizing enzyme facilitates a transient binding of said nucleotide to a nucleic acid strand complementary to said nucleic acid molecule, wherein said transient binding of said nucleotide having said particle coupled thereto effects said change in said electrical current, and wherein said nucleotide is incorporated into said nucleic acid strand subsequent to said transient binding; and (ii) use said change in said electrical current detected in (i) to identify said nucleotide, thereby identifying at least a portion of a sequence of said nucleic acid molecule.

In some embodiments, said polymerizing enzyme is contacted with a first buffer comprising a first cation to facilitate said transient binding of said nucleotide to said nucleic acid molecule; and wherein said polymerizing enzyme is subsequently contacted with a second buffer comprising a second cation to facilitate incorporation of said nucleotide into said nucleic acid strand. In some embodiments, said first cation is selected from the group consisting of Ca²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. In some embodiments, said second cation is selected from the group consisting of Mg²⁺, Mn²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. In some embodiments, said first cation is Ca²⁺ and said second cation is Mg²⁺.

In some embodiments, said polymerizing enzyme comprises a deoxyribonucleic acid (DNA) polymerase, a reverse transcriptase, an RNA-dependent RNA polymerase, an RNA polymerase, or any combination thereof. In some embodiments, said DNA polymerase is selected from the group consisting of Φ15, Φ29, BS32, B103, Cp-1, Cp-5, Cp-7, GA-1, G1, L17, M2Y, Nf, PRD1, PZE, PR4, PR5, PR722, PZA, SF5, T4, T7 DNA polymerases, or a functional variant thereof. In some embodiments, said polymerizing enzyme is a modified recombinant polymerase. In some embodiments, said polymerizing enzyme is modified such that it lacks 5′ to 3′ and/or 3′ to 5′ exonuclease activity.

In some embodiments, a binding kinetic of said nucleotide to said nucleic acid molecule is determined from said change in said electrical current, and wherein said binding kinetic is used to identify said nucleotide. In some embodiments, said binding kinetic is used to determine a modification state of a nucleotide of said nucleic acid molecule. In some embodiments, said modification state of nucleotide of said nucleic acid molecule comprises inosine, 5-methylcytosine, pseudouridine, N6-methyladenosine, N1-methyladenosine, 5′ cap-related 7-methylguanosine, and/or 2′-O-methylation.

In some embodiments, said solution is contained in a well, and wherein said well comprises a nanoelectrode of said plurality of electrodes.

Another aspect of the present disclosure provides a method for detecting a biomolecule, comprising: (a) providing a plurality of electrodes and a solution comprising a reversible redox moiety that is configured to facilitate redox cycling and to generate an electrical current between electrodes of said plurality of electrodes, wherein an individual electrode of said plurality of electrodes comprises a surface having a molecule comprising an affinity for said biomolecule coupled thereto; (b) directing said biomolecule to said molecule; (c) detecting a change in said electrical current using said plurality of electrodes upon said biomolecule coming into contact with said molecule; and (d) using said change in said electrical current detected in (c) to identify said biomolecule. In some embodiments, (c) comprises detecting said change in said electrical current using said plurality of electrodes upon binding of said biomolecule to said molecule.

In some embodiments, at least a portion of said solution is contained in a well, and wherein said well comprises said electrode of said plurality of electrodes. In some embodiments, said plurality of electrodes comprises an additional electrode external to said well. In some embodiments, said plurality of electrodes comprises a plurality of nanoelectrodes. In some embodiments, the method further comprises using a fluid flow unit configured to dispense said biomolecule. In some embodiments, said biomolecule comprises a protein or a polypeptide. In some embodiments, said biomolecule comprises a nucleic acid molecule.

In some embodiments, an additional electrode of said plurality of electrodes comprises an additional molecule, and wherein said change in said electrical current is detected upon an additional biomolecule coming into contact with said additional molecule. In some embodiments, said biomolecule comprises a particle coupled thereto, and wherein said additional biomolecule comprises an additional particle coupled thereto. In some embodiments, said biomolecule and said additional biomolecule are different, and wherein said particle and said additional particle have substantially identical sizes. In some embodiments, said biomolecule and said additional biomolecule are different, and wherein said particle and said additional particle have different sizes. In some embodiments, said biomolecule and said additional biomolecule are different, and wherein said particle and said additional particle have different surface areas. In some embodiments, said biomolecule and said additional biomolecule are different, and wherein said biomolecule and said additional biomolecule are coupled to said particle by tethers. In some embodiments, said tethers comprise a polymeric material. In some embodiments, said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof. In some embodiments, said tethers have different lengths.

In some embodiments, said particle comprises a plurality of polymers on a surface of said particle at a density such that said plurality of polymers reduces non-specific binding to said surface. In some embodiments, said plurality of polymers comprises a polymer selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof. In some embodiments, said particle and said additional particle comprise a plurality of polymers on surfaces at different densities. In some embodiments, said particle has a charge that electrostatically attracts said reversible redox moiety.

Another aspect of the present disclosure provides a system for biomolecule detection, comprising: a plurality of electrodes, wherein an individual electrode of said plurality of electrodes comprises a surface having a molecule specific for said biomolecule coupled thereto, wherein at least two of said plurality of electrodes are separated from each other by a gap, which gap is configured to receive at least part of a solution comprising a reversible redox moiety that is configured to facilitate redox cycling and to generate an electrical current between at least two of said plurality of electrodes; and a controller operatively coupled to said plurality of electrodes, which said controller is configured to (i) use said plurality of electrodes to detect a change in said electrical current upon said biomolecule being directed to and coming into contact with said molecule; and (ii) use said change in said electrical current detected in (i) to identify said biomolecule.

In some embodiments, said plurality of electrodes comprises a plurality of nanoelectrodes. In some embodiments, the system further comprises a well configured to contain at least part of said solution, wherein said well comprises said individual electrode of said plurality of electrodes. In some embodiments, said plurality of electrodes comprises an additional electrode external to said well. In some embodiments, the system further comprises a fluid flow unit configured to dispense said biomolecule. In some embodiments, said biomolecule comprises a protein or a polypeptide. In some embodiments, said biomolecule comprises a nucleic acid molecule. In some embodiments, said additional electrode comprises an additional molecule coupled to a surface of said additional electrode, wherein said well is configured to contain an additional biomolecule, wherein said change in said electrical current is detected upon said additional biomolecule coming into contact with said additional molecule.

In some embodiments, said biomolecule comprises a particle coupled thereto, and wherein said additional biomolecule comprises an additional particle coupled thereto. In some embodiments, said particle and said additional particle effect said change in said electrical current. In some embodiments, said biomolecule and said additional biomolecule are different, and wherein said particle and said additional particle have substantially identical sizes. In some embodiments, said biomolecule and said additional biomolecule are different, and wherein said particle and said additional particle have different sizes. In some embodiments, said biomolecule and said additional biomolecule are different, and wherein said particle and said additional particle have different surface areas.

In some embodiments, said biomolecule and said additional biomolecule are different, and wherein said biomolecule and said additional biomolecule are coupled to said particle and said additional particle via tethers. In some embodiments, said tethers comprise a polymeric material. In some embodiments, said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof. In some embodiments, said tethers have different lengths.

In some embodiments, said particle comprises a plurality of polymers on a surface of said particle at a density configured to reduce non-specific binding to said surface. In some embodiments, said plurality of polymers comprises a polymer selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof. In some embodiments, said particle and said additional particle comprise a plurality of polymers on surfaces at different densities. In some embodiments, said particle has a charge that electrostatically attracts said reversible redox moiety.

Another aspect of the present disclosure provides a non-transitory computer readable medium comprising machine executable code that, upon execution by one or more computer processors, can implement any of the methods above or elsewhere herein.

Another aspect of the present disclosure can provide a system comprising one or more computer processors and computer memory coupled thereto. The computer memory can comprise machine executable code that, upon execution by one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an experimental result of a redox cycling performed on a sensor chip comprising nanoelectrodes;

FIG. 2 shows a schematic view of a manufacturing process of coplanar electrodes;

FIG. 3 shows a schematic view of initial parts of a manufacturing process for creating a nano-gap using a sacrificial material;

FIG. 4 shows a schematic view of later parts of the manufacturing process shown in FIG. 3;

FIGS. 5A-5B show schematic views of a non-coplanar sensor design with a redox cycling modifying particle and COMSOL MULTIPHYSICS® (COMSOL) modeling results using the sensor. FIG. 5A shows a perspective view of a cross-section of the sensor and a COMSOL modeling results with the use of a redox cycling modifying particle. FIG. 5B shows a cross-sectional view of the sensor and a COMSOL modeling result with the use of a large redox cycling modifying particle;

FIG. 6 shows a schematic view of the deoxyribonucleic acid (DNA) sequencing method using two different sized redox cycling modifying particles;

FIG. 7 shows a schematic of a redox cycling modifying particle with a coating to prevent non-specific binding;

FIG. 8 shows a schematic view of a possible fluidic interface of a sensor chip;

FIG. 9 shows a schematic view of a process to capture epigenetic information;

FIG. 10 shows a schematic view of a synthetic nanopore with a nanoelectrode pair;

FIG. 11 shows a COMSOL modeling result of redox cycling current with particles at different heights off the surface. with a nanoelectrode sensor.

FIG. 12 shows a computer system that is programmed or otherwise configured to implement methods and systems provided herein.

FIG. 13 shows data showing pulses from 100-nanometer (nm) diameter polystyrene nanospheres freely diffusing into 200-nm diameter sensor wells.

FIG. 14 shows a histogram of current blocking current levels from three runs each with a different diameter polystyrene nanosphere diffusing into 200-nm diameter sensor wells.

FIG. 15A-15B show SEM images of 200-nm diameter sensor chips. FIG. 15A shows an example wherein an area of the top level electrode is larger than that of the bottom electrode. FIG. 15B shows an SEM image of the chip with inadequate control of non-specific binding.

FIG. 16 shows a schematic of an example two-chip system.

FIG. 17A-17B shows a cross section of an example sensor fabrication method.

FIG. 18 shows a schematic illustration of an example current trace from a 4-label synchronous system.

FIG. 19 shows a schematic illustration of an example current trace from a 2-label synchronous system.

FIG. 20 shows a schematic illustration of an example current trace from a fast 2-label synchronous system.

FIG. 21 shows an example viewing window of a computer simulation of a 4-label free running system where all 4 bases are in solution and the bases are added at the speed of the polymerase.

FIG. 22 shows an example confusion matrix output from a computer simulation with a simple base identification routine.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “adjacent” or “adjacent to,” as used herein, includes ‘next to’, ‘adjoining’, ‘in contact with’, and ‘in proximity to’. In some instances, adjacent to components are separated from one another by a certain distance. “Adjacent” may denote proximity between two or more objects. “Adjacent” may comprise physical contact. “Adjacent” may comprise physical separation. “Adjacent” may signify that two objects are within sufficient proximity to interact directly or indirectly through a third object, for example through a bead coupled to a nucleotide.

The term “biomolecule,” as used herein, generally refers to any biological molecule or material (e.g., a cell, an enzyme, an enzyme substrate, etc.). The biomolecule may be a biopolymer (e.g., a biological molecule having subunits, such as repeating subunits). The biomolecule may be a nucleic acid molecule, protein, lipid, or carbohydrate. The biomolecule may include one or more subunits, such as nucleotides or amino acids. The biomolecule can be a ribonucleic acid (RNA) molecule, a deoxyribonucleic acid (DNA) molecule, or a combination or variant thereof.

The term “interrogated base,” as used herein, generally refers to a nucleic acid base which is being interrogated, such as by sequencing. The interrogated base may be a first base which may not have a complementary base incorporated as a part of an extended primer, and which may be complementary to the next base which can be incorporated by a polymerase, a reverse transcriptase or another enzyme. A base being incorporated may be one base in the 5′ direction from the last base which has a complementary base which is a part of an extended primer, and which may further be bound at the active site of an enzyme such as a polymerase.

The term “electrode,” as used herein, generally refers to a conductor through which electrons enter or leave a fluid. An electrode (or electrode part) can be used to measure electrical current to or from another electrode. The electrodes can be used to measure an electrical signal (e.g., current). The electrode may be a nanoelectrode (e.g., an electrode comprising a dimension less than or equal to about 1 micron). The electrode may be part of an electrode pair. An electrode pair may be separated by a gap, such as a nano-gap (e.g., a gap having a dimension less than or equal to about 1 micron). An electrode pair may comprise a nanoelectrode. An electrode pair may comprise two nanoelectrodes. In some cases, the electric current can be generated through redox cycling. The electrodes can be used to measure a change in the electric current associated with a molecular binding event occurring within or near the nanogap. The change may be an increase or a decrease in the electric current.

The term “gap,” as used herein, generally refers to a well, pore, channel, space, or passage formed or otherwise provided. A gap may be a nanogap (e.g., may comprise a dimension less than or equal to about 1 micron). In some cases, the gap is where the majority of the redox cycling of the redox species occurs. Electrodes may be located within, across, adjacent to, or in proximity of the gap or any combination thereof. The gap may be between two electrodes. The gap may be coupled to a sensing circuit. In some examples, a gap has a characteristic width or diameter on the order of 1 nanometer (nm) to about 1000 nm. A gap having a dimension (e.g., a width) on the order of nanometers may be referred to as a “nano-gap” (also “nanogap” herein). In some situations, a nano-gap has a dimension that is from about 1 nanometer (nm) to 200 nm, 10 nm to 150 nm, 5 nm to 100 nm, 10 nm to 200 nm, or 20 nm to 250 nm, or no greater than 200 nm, 100 nm, 90 nm, 80 nm, 70 nm, 60 nm, 50 nm, or less. In some cases, a nano-gap has a dimension that is less than or equal to about 1,000 nm, 900 nm, 800 nm, 700 nm, 600 nm, 500 nm, 400 nm, 300 nm, 200 nm, 150 nm, 100 nm, 50 nm, 20 nm, 10 nm or less. The nano-gap can have a dimension at least about 5 nm, 6 nm, 7 nm, 8 nm, 9 nm, 10 nm, 20 nm, 30 nm, 40 nm, 50 nm or more. In some cases, the width of a nano-gap can be less than the diameter of a biomolecule or a subunit (e.g., monomer) of the biomolecule. Nano-gap may be filled with a solution containing a reversible redox moiety.

The term “particle,” as used herein, generally refers to any entity having a diameter or a size of less than 10 microns (μm). In some cases, the particle is a nanoparticle which has a characteristic dimension (e.g., a diameter) between about 1 nm and about 1,000 nm. In some cases, the particle has a characteristic dimension between about 50 nm and 100 nm. In some cases, the particle has a characteristic dimension between about 20 nm and 50 nm. In some cases, the particle has a characteristic dimension between about 30 and 80 nm. In some cases, the particle has a characteristic dimension between about 80 and 150 nm. In some cases, the particle has a characteristic dimension between about 100 and 200 nm. In some cases, the particle has a characteristic dimension between about 100 and 300 nm. In some cases, the particle has a characteristic dimension between about 200 and 500 nm. In some cases, the particle has a characteristic dimension between about 500 and 1000 nm (1 μm). In some cases, the particle has a characteristic dimension between about 1 μm and 3 μm. In some cases, the particle has a characteristic dimension between about 1 μm and 10 μm. In some cases, the particle has a characteristic dimension that is about 80% of a diameter of a cavity comprising an electrode pair. In some cases, the particle has a characteristic dimension that is about 60% of a diameter of a cavity comprising an electrode pair. In some cases, the particle has a characteristic dimension that is about 40% of a diameter of a cavity comprising an electrode pair. In some cases, the particle has a characteristic dimension that is about 20% of a diameter of a cavity comprising an electrode pair. In some cases, the particle has a characteristic dimension that is less than 20% of a diameter of a cavity comprising an electrode pair. In some cases, a smaller particle-to-cavity diameter ratio may favor faster or more frequent diffusion of the particle into the cavity. In some cases, a larger particle may affect a larger change in a redox cycling current between an electrode pair disposed within a cavity. In some cases, the particle has a characteristic dimension that is approximately equal to or greater in size than a dimension (e.g., the diameter of the cavity) of the cavity. In this case, the particle may only partially enter the cavity. The characteristic dimension of the particle may be a diameter. In some cases, a particle is spherical or approximately spherical. For example, a particle may comprise an approximately ellipsoidal shape with at most a 50%, 40%, 30%, 20%, 10%, 5%, 2%, or 1% difference in the lengths of its largest and smallest axes.

The particle can comprise latex, silica, polymeric materials such as polystyrene or poly(methyl methacrylate) (PMMA), metallic materials (e.g., gold, silver, platinum, cobalt, or the like), ceramic materials, semiconductor, dendrimeric particles, oxides such as silicon dioxide, or composites or combinations thereof. In cases where the particles comprise polymeric materials, the polymers may be branched, partially branched, unbranched, cross-linked, or partially cross-linked. The particle may or may not be functionalized. The particle may be solid or porous. The particle may be of any shape, regular or irregular. The particle may comprise a spherical shape, spheroidal shape, ellipsoidal shape, non-spherical shape, box-like shape, rod-like shape, or any distorted version thereof (e.g., comprising a surface feature such as a bump or a ridge). A particle may be spherical or substantially spherical. The particle may be a bead. As provided herein, the particle may modify the magnitude of redox cycling current. The particle may include a coating(s) which may reduce non-specific binding, increase the effective diameter of the particle, reduce agglomeration, enhance wetting, or include functional compounds or moieties such as tethers. A particle may comprise more than one material. For example, a particle may comprise a core comprising a first material, and a coating comprising a second material. For example, a particle may comprise a magnetic metal oxide core and a non-magnetic polymer coating. A particle may be present at a concentration of about 0.1 nM to 0.5 nM, about 0.5 nM to 1 nM, about 1 nM to 5 nM, about 5 nM to 10 nM, about 10 nM to 20 nM, about 20 nM to 40 nM, about 30 nM to 50 nM, about 40 nM to 80 nM, about 50 nM to 100 nM, about 80 nM to 150 nM, or about 100 nM to 200 nM.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or analogs or variants thereof. A nucleotide can include any subunit that can be incorporated into a growing nucleic acid strand. A nucleotide may denote a biomolecule with a nucleotide-unit capable of incorporating into a nucleic acid (e.g., a nucleoside triphosphate). Variants of nucleotides may include for example, but not limited to: de-novo methylation, maintenance methylation, demethylation; methylation, ubiquitylation, phosphorylation, sumoylation, monomethylation, dimethylation, trimethylation, and the like. A nucleotide variant may comprise a modification (e.g., a methyl group) on a nucleobase, on a sugar (e.g., a ribosyl moiety), or on a phosphoryl (e.g., a phosphoryl of a triphosphate moiety). Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is a deoxyribonucleic acid (DNA) or ribonucleic acid (RNA), or derivatives thereof. A nucleic acid may be single-stranded or double-stranded.

The term “polypeptide,” or “peptide,” as used herein, generally refers to a biological molecule, or macromolecule, having amino acid monomers, subunits or residues. The polypeptide may be a protein (e.g., in cases in which there are secondary and/or tertiary structures). The amino acid monomers can be selected from any naturally occurring and/or synthesized amino acid monomer, such as, for example, 20, 21, or 22 naturally occurring amino acids. In some cases, 20 amino acids are encoded in the genetic code of a subject. Some polypeptides may include amino acids selected from about 500 naturally and non-naturally occurring amino acids. In some situations, a polypeptide can include one or more amino acids selected from isoleucine, leucine, lysine, methionine, phenylalanine, threonine, tryptophan and valine, arginine, histidine, alanine, asparagine, aspartic acid, cysteine, glutamine, glutamic acid, glycine, proline, serine and tyrosine.

The present disclosure provides a chip with electrode sensors, or a set of chips, to sequence a biomolecule (e.g., a nucleic acid molecule). The chip can be upscaled to comprise a plurality of sensors (e.g., greater than or equal to about 1 million, 5 million, 10 million, 20 million, 30 million, 40 million, 50 million, 60 million, 70 million, 80 million. 90 million, 100 million sensors, or more). The sensor chip may be cleaned and reused multiple times enabling even lower sequencing costs.

Due to the large amount of sequence information in a genome, a method that provides massively parallel, low cost detection is desirable. Integration of >100 million (M) sensors onto a silicon chip can facilitate massively parallel detection. However, creating a detectable current level can require a large number of electrons in an electrochemical sensor. For example, 1 pA is 6,244,000 electrons/sec.

One way to create a large number of electrons in an electrochemical sensor can be to use redox cycling using a reversible redox compound in solution. When an appropriate voltage is applied between the nanoelectrodes the reversible redox compound can be oxidized at one electrode, diffuse to the other electrode and be reduced. This process can transfer one or more electrons per round trip.

### Redox Cycling

The present disclosure provides systems and methods that can be used to detect or identify a biomolecule, such as a nucleic acid molecule. The biomolecule can be detected or identified using one or more pairs of electrodes by measuring a change in the electrical current. An electrode pair may generate a baseline electric current when held at a potential. The baseline electric current may be about 100-200 picoamperes (pA). The baseline electric current may be about 1 pA to 10 pA. The baseline electric current may be about 10 pA to 20 pA The baseline electric current may be about 20 pA to 50 pA. The baseline electric current may be about 50 pA to 100 pA. The baseline electric current may be about 50 pA to 200 pA. The baseline electric current may be about 50 pA to 300 pA. The baseline electric current may be about 100 pA to 300 pA. The baseline electric current may be about 100 pA to 400 pA. The baseline electric current may be about 200 pA to 500 pA. The baseline electric current may be about 400 pA to 1000 pA (1 nanoampere, nA). The baseline electric current may be about 500 pA to 1.5 nA. The baseline electric current may be about 1 nA to 2 nA. The baseline electric current may be about 1 nA to 3 nA. The baseline electric current may be about 2 nA to 5 nA. The baseline electric current may be about 4 nA to 10 nA. The baseline current may be stable over a period of time. For example, an average baseline current level (e.g., for an electrode pair or for a plurality of electrode pairs) may change by less than 3% per minute. An average baseline current level may change by less than 2% per minute. An average baseline current level may change by less than 1% per minute. An average baseline current level may change by less than 0.5% per minute. An average baseline current level may change by less than 0.25% per minute. An average baseline current level may change by less than 0.1% per minute. An average baseline current level may change by less than 0.05% per minute. An average baseline current level may change by less than 0.02% per minute.

The amplitude of the electrical current may increase as the gap between the electrodes is narrowed. The electrodes may be closely spaced. The electrodes may be spaced apart from one another by a gap. The gap may be a nanogap which has the longest dimension less than or equal to about 1,000 nm. The gap may be a nanogap which has the longest dimension less than or equal to about 750 nm. The gap may be a nanogap which has the longest dimension less than or equal to about 500 nm. The gap may be a nanogap which has the longest dimension less than or equal to about 300 nm. The gap may be a nanogap which has the longest dimension less than or equal to about 200 nm. The gap may be a nanogap which has the longest dimension less than or equal to about 100 nm. The gap may be a nanogap which has the longest dimension less than or equal to about 80 nm. The gap may be a nanogap which has the longest dimension less than or equal to about 60 nm. The gap may be a nanogap which has the longest dimension less than or equal to about 50 nm. The gap may be a nanogap which has the longest dimension less than or equal to about 40 nm. The gap may be a nanogap which has the longest dimension less than or equal to about 30 nm. The gap may be a nanogap which has the longest dimension less than or equal to about 25 nm. The gap may be a nanogap which has the longest dimension less than or equal to about 20 nm. The gap may be a nanogap which has the longest dimension less than or equal to about 15 nm. The gap may be a nanogap which has the longest dimension less than or equal to about 10 nm. The gap may be a nanogap which has the longest dimension between about 5 nm and 20 nm. The gap may be a nanogap which has the longest dimension between about 20 nm and 40 nm. The gap may be a nanogap which has the longest dimension between about 40 nm and 80 nm. The gap may be a nanogap which has the longest dimension between about 80 nm and 200 nm. The gap may be a nanogap which has the longest dimension between about 100 nm and 300 nm. The gap may be a nanogap which has the longest dimension between about 250 nm and 500 nm. The gap may be a nanogap which has the longest dimension between about 400 nm and 800 nm. The gap may comprise a depth of at most 1000 nm. The gap may comprise a depth of at most 750 nm. The gap may comprise a depth of at most 500 nm. The gap may comprise a depth of at most 300 nm. The gap may comprise a depth of at most 200 nm. The gap may comprise a depth of at most 150 nm. The gap may comprise a depth of at most 100 nm. The gap may comprise a depth of at most 60 nm. The gap may comprise a depth of at most 40 nm. The gap may comprise a depth of about 30 nm. The gap may comprise a depth of at most 25 nm. The ratio of depth to width may affect the residence time and frequency of particle diffusion into a gap. A gap may comprise a greater depth than width. A gap may comprise a greater width than depth. A gap may comprise a depth and a width of approximately equal size.

The redox cycling may be a process that allows one or more redox-active moieties (such as reversible redox compounds or moieties) to diffuse back and forth rapidly (e.g., less than or equal to about lmillisecond (ms), 0.8 ms, 0.6 ms, 0.5 ms, 0.4 ms, 0.3 ms, 0.1 ms, 0.03 ms, 0.01 ms, 0.003 ms, 0.001 ms, 0.0003 ms, 0.0001 ms, or less for one cycle) between two or more electrodes. The redox moiety may contact (e.g., physically contact or come within sufficient proximity of the electrode to accept or transfer an electron through an outer-sphere electron transfer process, such as an electron transfer while the redox moiety and electrode are separated by 0.5 nm). When appropriate voltages are applied to the nanoelectrodes, the reversible redox moiety can be oxidized on one electrode and diffuse across the nano-gap to the other electrode to be reduced. This process can transfer one or more electrons per cycle. The nano-gap can contain one or more of the reversible redox moieties during the redox cycling. The redox moieties can be present in a concentration of less than 100 mM, 30 mM, 10 mM, 3 mM, 1 mM, 0.3 mM, 0.1 mM, or less. Each of the reversible redox moieties can diffuse between the nanoelectrodes multiple times (e.g., over a thousand times) each second, generating a measurable redox cycling current. The measurable redox cycling current may provide a baseline current. The baseline current may be stable over seconds, minutes, or hours in the absence of a change in conditions, such as a change in concentration of a redox moiety or the entry of a particle into the gap between an electrode pair (e.g., a nano-gap). The reversible redox moieties can be selected from a group including, but not limited to, Hexaammineruthenium(III) chloride, methyl viologen dichloride, hexacyanoferrate(II)trihydrate, ferricyanide, 4-aminophenyl, methylene blue, alloxan, ions of manganese, phenazine methosulfate, menadione, copper/putrescine/pyridine, paraquat, doxorubicin, bleomycin, and ruthenium (II) tris-(1,10-phenanthroline-5,6-dione), ferrocene compounds such as ferrocenedimethanol, 1,1′-Ferrocenedicarboxylic acid or (Ferrocenylmethyl)trimethylammonium Chloride, or mixtures thereof. The reversible redox moieties may be selected to minimize the inhibition of nearby enzymes such as polymerase. The concentration of reversible redox moieties may be low enough to reduce the inhibition of the enzymes.

The change in electrical current can be brought on by the presence of one or more particles (e.g., one or more redox cycling modifying particles) in the electrode gap (e.g., an electrode nano-gap). A particle, such as a nanoparticle (or a macromolecule such as protein), that diffuses or is guided into the nano-gap may alter the redox cycling process (e.g., efficiency, rate). Such alteration may produce a measurable change in the redox cycling current (e.g., the baseline electric current). This can occur as a current decrease or a current increase. A current decrease may occur when a particle impedes the diffusion of reversible redox molecules between the nanoelectrode pairs or occludes the electrode surface causing a decrease in redox cycling efficiency. A current increase may occur when the presence of the particle between the electrodes, for example, increases the local concentration of reversible redox molecules. A current increase may also occur when the particle is conductive (e.g., comprises a conductive polymer coating).

The change in electrical current caused by a particle may depend on the size of the particle. The change may be a decrease in electrical current. The change in electrical current may correlate with the size of the particle. The change in electrical current caused by a particle may comprise a proportionality to the particle's size. The change in electrical current caused by a particle may comprise a proportionality to a ratio of particle and electrode gap dimensions. The change in electrical current may be about 0.1 pA to 0.5 pA, about 0.5 pA to 1 pA, about 1 pA to 5 pA, about 3 pA to 10 pA, about 5 pA to 15 pA, about 10 pA to 20 pA, about 20 pA to 40 pA, about 40 pA to 60 pA, about 60 pA to 80 pA, about 80 pA to 100 pA, about 100 pA to 120 pA, about 120 pA to 150 pA, about 150 pA to 200 pA, about 200 pA to 300 pA, about 300 pA to 500 pA, or about 500 pA to 1000 pA. In many instances, the change in electrical current is proportional to the baseline electrical current. The change in electrical current may be about 0.1% to 0.5%, about 0.5% to 1%, about 1% to 2%, about 2% to 4%, about 3% to 5%, about 2% to 6%, about 4% to 8%, about 5% to 10%, about 8% to 12%, about 10% to 15%, about 10% to 20%, about 20% to 30%, or about 20% to 40% of the baseline electrical current. The change in electrical current may be about 0.1%, about 0.5%, about 1%, about 2%, about 3%, about 4%, about 5%, about 8%, about 10%, about 12%, about 15%, about 20%, about 25%, about 30%, about 35%, about 40%, or greater than about 40% of the baseline electrical current. For example, a nanoelectrode pair comprising a baseline electrical current of about 200 pA may exhibit an electrical current decrease of 20 pA (10%) upon diffusion of a particle (e.g., a non-conductive nanoparticle) into a nanogap between the nanoelectrode pair.

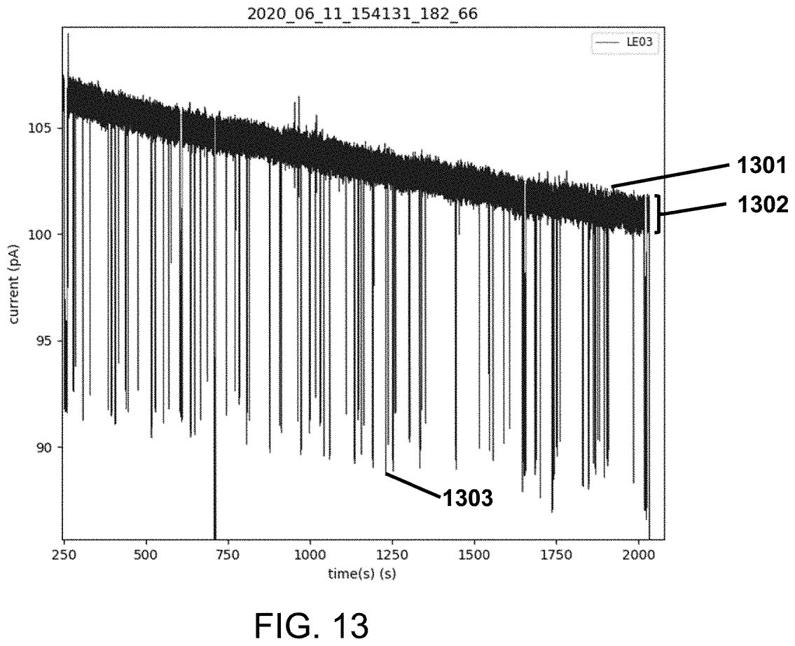

In some cases, the change in electrical current caused by a particle is larger than the background noise of the electrical current. For example, a nanoelectrode pair comprising a baseline electrical current of about 100 pA and a noise (stdev of 0.65 pA) may exhibit electrical current decreases of about 14 pA upon diffusion of a particle into a nanogap between the electrode pair, thus exhibiting a signal-to-noise ratio (signal mean/stdev) of about 21.5 to 1. An example of such a system is provided in FIG. 13, which depicts a 200 nm electrode gap held at a 0.6V difference in the presence of 10 mM ferrocene dimethanol and 100 nm polystyrene particles in a 100 mM phosphate buffer. In this example, the baseline current 1301 is about 105 pA and a corresponding noise level 1302 of about 2 pA p-p or a stdev of ˜0.65 pA. The diffusion of 100 nm polystyrene particles affects current decreases 1303 of about 15 pA, corresponding to a signal-to-noise level of about 23 to 1. The systems disclosed herein may exhibit signal-to-noise levels of at least 2-to-1, at least 3-to-1, at least 4-to-1, at least 5-to-1, at least 8-to-1, at least 10-to-1, at least 12-to-1, at least 15-to-1, at least 20-to-1, at least 25-to-1, at least 30-to-1, at least 35-to-1, at least 40-to-1, or at least 50-to-1. The systems disclosed herein may comprise a noise level of at most about 0.1 pA, at most about 0.2 pA, at most about 0.3 pA, at most about 0.4 pA, at most about 0.5 pA, at most about 0.6 pA, at most about 0.8 pA, at most about 1 pA, at most about 1.2 pA, at most about 1.5 pA, at most about 2 pA, at most about 2.5 pA, at most about 3 pA, at most about 4 pA, at most about 5 pA, at most about 6 pA, at most about 8 pA, at most about 10 pA, at most about 12 pA, at most about 15 pA, at most about 20 pA, or at most about 25 pA.

The change in the redox cycling current can be utilized to detect single-molecule binding events. In some cases, a biomolecule of interest may be attached to the redox cycling modifying particle that can cause a predetermined amount of change in the current. If the nanoelectrodes measure the predetermined amount of change in the current, the presence of the biomolecule can be detected. In some cases, a redox cycling modifying particle which can cause a predetermined amount of current change may be used as a reference. The predetermined amount of the current change may be used as a value to calibrate or normalize measured current change caused by other particles.

The present disclosure provides a method for detecting a biomolecule. The method may comprise the step of providing a plurality of electrodes (e.g., nanoelectrodes) and a solution comprising one or more reagents. For example, the solution may comprise a redox-active molecule or moiety (e.g., a reversible redox molecule or moiety). The redox-active molecule may facilitate or be capable of redox cycling which may generate an electrical current in the plurality of electrodes. The method may also comprise directing the biomolecule having a particle coupled thereto to the solution. The particle may be a redox cycling current modifying particle. The particle (e.g., a redox cycling current modifying particle) may be a nanoparticle as described above or elsewhere herein. The particle may cause a change in the electrical current detected by the plurality of electrodes. The change in the electrical current detected may be used to identify the biomolecule.

Also provided in the present disclosure is a system for biomolecule detection, which system may comprise a plurality of electrodes such as a plurality of nanoelectrodes. The system may also comprise a solution. In some cases, the system is configured to receive a solution and bring at least a part thereof in contact with at least a part of the plurality of the electrodes. The solution may comprise one or more redox-active molecules or moieties such as a reversible redox molecule or moiety. The redox-active molecules may facilitate or be capable of redox cycling which may generate an electrical current in the plurality of electrodes. The redox cycling may comprise electrochemical-electrochemical redox cycling which may comprise oxidation and reduction occurring in two electrodes, electrochemical-chemical redox cycling which may comprise the use of one electrode and one reductant, enzymatic-enzymatic redox cycling which may comprise the use of one reductant and one oxidant, or any other types of redox cycling which may comprise the use of a redox-active molecule(s) or moiety(ies), or a combination or variation thereof.

At least two electrodes may be separated from each other by a gap. The term “facilitates redox cycling” may refer to conditions where electrodes may be spaced less than or equal to about 1 micrometer (μm) such that one or more redox-active molecule(s) or moiety(ies) may travel rapidly between and be oxidized and reduced. The movement of the redox-active molecule(s) or moiety(ies) between the closely-spaced electrodes may generate an electrical signal or effect a change in an electrical signal which may be detected or measured by the electrodes.

The size of the electrical signal or the change in the electrical signal may depend on a number of factors. The size of the electrical signal or signal change may in part depend on electrode design. In many instances, electrical signal size comprises a proportionality to electrode surface area, redox solution concentration, viscosity, etc.

Sample conditions may also affect the size of the electrical signal or the change in the electrical signal. In many cases, the size of the electrical signal or the change in the electrical signal may be increased by raising the concentration of redox-active molecules. The size of the electrical signal or the change in the electrical signal may comprise a dependence on redox-active molecule type. For example, selecting a faster diffusing redox-active molecule may generate a larger electrical signal or change in electrical signal. In many cases, the size of the electrical signal or the change in the electrical signal may be adjusted by changing the temperature of the system.

Thus, the present systems and methods provide control over electrical signal size. In some cases, an electrical signal is around 100 pA. In some cases, an electrical signal is between about 1 pA and 10 pA. In some cases, an electrical signal is between about 10 pA and 20 pA. In some cases, an electrical signal is between about 20 pA and 50 pA. In some cases, an electrical signal is between about 50 pA and 100 pA. In some cases, an electrical signal is between about 50 pA and 200 pA. In some cases, an electrical signal is between about 100 pA and 200 pA. In some cases, an electrical signal is between about 100 pA and 300 pA. In some cases, an electrical signal is between about 100 pA and 400 pA. In some cases, an electrical signal is between about 200 pA and 500 pA. In some cases, an electrical signal is between about 400 pA and 1000 pA (1 nA). In some cases, an electrical signal is between about 500 pA and 1.5 nA. In some cases, an electrical signal is between about 1 nA and 2 nA. In some cases, an electrical signal is between about 1 nA and 3 nA. In some cases, an electrical signal is between about 2 nA and 5 nA. In some cases, an electrical signal is between about 4 nA and 10 nA.

In a system comprising multiple electrode pairs, a first electrode pair may comprise a first electrical signal with a first size and a second electrode pair may comprise a second electrical signal with a second size. In some cases, the first electrical signal may comprise a higher signal-to-noise ratio than the second electrical signal, and the second electrical signal may comprise a larger signal Conversely, a plurality of electrode pairs may comprise electrical signals of about the same size (e.g., within 50%, 20%, 10%, 5%, 2%, 1%, 0.5%, 0.2%, or 0.1%)

The system may further comprise a controller operatively coupled to the plurality of electrodes, in which the controller may be configured to use the plurality of electrodes to detect the electrical current and/or changes thereof when a biomolecule is directed to the solution. The biomolecule may comprise a particle coupled thereto. The particle may be a nanoparticle. The particle (e.g., the nanoparticle) may be a redox cycling current modifying particle. The particle may affect the change in the electrical current and/or a change thereof. The system may further comprise, be connected to, or be configured to be connected to, a CPU or a computing system, which can use the electrical current detected or changes thereof to identify the biomolecule.

In some cases, an electrode of the plurality of electrodes comprises a molecule which is coupled to a surface of the electrode. The electrode may be a nanoelectrode. The molecule may be capable of binding the biomolecule. The molecule may be configured to specifically bind to the biomolecule. The molecule may be attached to the surface directly or indirectly (e.g., through a linker). The molecule may bind to the biomolecule when the biomolecule is directed to and comes into contact with the molecule. The binding may be transient or permanent. Upon binding of the biomolecule to the molecule, the biomolecule may inhibit or block at least a portion of the reversible redox molecules diffusing between the plurality of the electrodes, thereby affecting the electric current detected by the electrodes. The molecule may be a protein, a polypeptide, a nucleic acid molecule, an antigen, or any type of molecular recognition entity or moiety.

In some cases, the plurality of electrodes comprises a plurality of molecules coupled to a surface of an individual electrode of the plurality of the electrodes. The plurality of molecules may comprise molecules that are the same or of different types. Each of the molecules may be configured to bind to a specific type of biomolecule, or a different molecule of the same type (e.g., a wild-type gene and its mutants). Binding events associated with each molecule may be independently or individually detected. Multiple binding events may be simultaneously detected, which may enable multiplexed detection of a plurality of biomolecules.

The redox cycling detection system and method can be used to detect single-molecule binding events. A biomolecule of interest may be attached to a particle (e.g., a redox cycling current modifying particle). If no binding occurs between the biomolecule attached to the particle and a molecule in the nano-gap, then the particle can rapidly diffuse out of the nano-gap. However, if the particle binds to a molecule in the nanogap area, the presence of the particle can cause a sustained and measurable change in redox cycling. The resulting change in electric current (e.g., redox cycling electric current) can be measured by the nanoelectrodes to allow the detection of the binding event. A fluid flow unit may be configured to dispense the biomolecule with the attached particle.

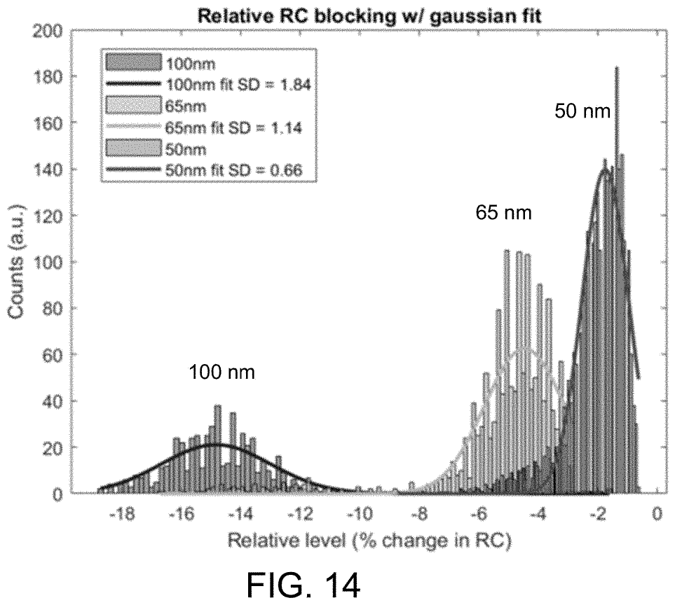

A system may comprise multiple particles comprising different biomolecules or combinations of biomolecules. For example, a system comprises a first particle attached to adenosine triphosphate (ATP), a second particle attached to guanosine triphosphate (GTP), a third particle attached to thymidine triphosphate (TTP), a fourth particle attached to cytidine triphosphate (CTP), and a polymerase enzyme in an electrode gap. Multiple particles may each generate a different signal upon diffusion into an electrode gap, thereby allowing different particle diffusion or binding (e.g., binding to a protein in or near an electrode nanogap) events. The multiple particles may comprise different sizes, and thereby generate different changes in the electrical current of an electrode pair upon diffusion between the electrode pair. An example of such a system is provided in FIG. 14, which summarizes the current changes caused by the diffusion of 100 nm, 65 nm, and 50 nm particles into a 200 nm gap between an electrode pair. The particles can be distinguished by the sizes of the current changes corresponding to their diffusion into the gap between the electrode pair. The 100 nm particle generated an average change in electrical current of 15%, while the 65 nm particle generated an average change in electrical current of 5%, and the 50 nm particle generated an average change in electrical current of about 2%. Notably, each particle generated a range of electrical current changes following Gaussian-like distributions. While the electrical current changes generated by the 100 nm particle negligibly overlapped the electrical current changes generated by the 65 nm and 50 nm particles, the current changes generated by the 65 nm particle partially overlapped the current changes generated by the 50 nm particle.

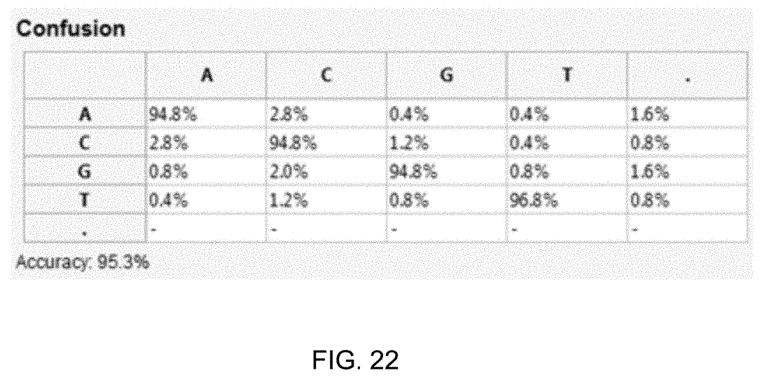

The present disclosure strategies for diminishing signal overlap from different particle types, thus increasing the accuracy of particle identifications. In some cases, increasing electrode gap depth, optimizing linker length, or immobilizing a particle-binding molecule at the bottom of an electrode gap may prevent a particle from only partially entering the electrode gap, thereby decreasing signal size variation. In some cases, a signal change may be averaged over a sufficiently long timeframe to allow signals from different particles to be distinguished. For example, a system may provide low activity polymerases capable of holding nucleoside triphosphates without performing cleavage, and thereby enable nucleoside triphosphate detection with second or minute time lengths. In some cases, two or more particles may generate sufficiently different signals such that each can be accurately identified. A system or method may comprise at least 85%, at least 90%, at least 92%, at least 94%, at least 95%, at least 96%, at least 97%, at least 98%, at least 99% or greater than 99% accuracy (e.g., percent of identifications that are correct identifications). In some instances, a system or method may comprise an accuracy of at least 95%. In some instances, a system or method may comprise an accuracy of at least 98%. For example, FIG. 22 provides an estimated raw accuracy of 95.3% for a four-particle system for nucleic acid sequencing. Because the errors can be mostly stochastic, consensus accuracy for the systems and methods disclosed herein can be very high.

Another strategy for diminishing signal overlap may comprise attaching more than one biomolecule of interest to a specific particle size, thereby lowering the number of particles required for an assay or experiment. For example, a system may comprise a first particle size attached to ATP and the same particle size attached to GTP, and a second larger particle size attached to TTP and the second particle size attached to CTP. Different biomolecules and particles may also be introduced to a system at different times. A method may comprise contacting a system with a first particle, removing the first particle from the system (e.g., by flushing the system with a particle free solution), and then contacting the system with a second particle. A method may also comprise contacting a system with a first particle and a second particle, removing the first and second particle types, and contacting the system with a third and fourth particle type. For example, two solutions could be created, each with a small and a larger particle (e.g. a first solution of small particle ATP and large particle CTP labeled particles and a second solution of small particle GTP and large particle TTP labeled particles). When the first solution is delivered, and the particle is captured in the well by the polymerase, a small pulse may indicate binding of ATP and a larger pulse may indicate binding of CTP. Similarly, when the second solution is delivered, a small pulse may indicate binding of TTP and a larger pulse may indicate binding GTP. No pulse may mean that the base was not complementary to either of the two nucleotide labeled beads.

To avoid overlap entirely, a single particle size could be used for each nucleotide. They would then be delivered one nucleotide type at a time and a pulse would indicate the base was complementary to the nucleotide delivered. This method may require more deliveries and therefore may be slower. A method may also comprise contacting the system with a non-particle associated biomolecule. In such cases, the non-particle associated biomolecule and a particle associated biomolecule may be introduced to a system at different points in time. For example, a system may be contacted by a first particle attached to ATP a second particle attached to GTP, and a third particle attached to TTP, washed, and then contacted with particle-free CTP, such that incorporations of ATP, GTP, and TTP by a polymerase provide signals, while incorporation of CTP does not generate a signal.

The biomolecule of interest can be a protein or polypeptide. The biomolecule can be a nucleic acid. The methods disclosed herein can be used to sequence nucleic acids since the nanoelectrodes can detect an individual binding event. The present disclosure provides a method for analyzing or detecting a nucleic acid molecule by providing a plurality of nanoelectrodes and a solution. The solution may comprise a molecular recognition entity such as a nucleic acid molecule having a polymerizing enzyme coupled thereto. As an example, the molecular recognition entity may be a polymerizing enzyme coupled (e.g., transiently or stably bound) to a primed nucleic acid template. In some cases, the molecular recognition entity is the nucleic acid molecule to be analyzed or detected. The nucleic acid molecule may be self-primed. For example, the nucleic acid may have a universal primer and its complement linked together via a hairpin or tether. The nucleic acid molecule may comprise a polymerizing enzyme coupled thereto. The solution may also comprise one or more redox-active molecules or moieties, e.g., a reversible redox moiety that facilitates or is capable of redox cycling to generate an electrical current in the plurality of nanoelectrodes. Subsequently, the nucleic acid molecule may be brought into contact with a nucleotide having a particle coupled thereto, under conditions such that the nucleotide binds to a nucleic acid strand (e.g., a primer strand) complementary to the nucleic acid molecule with the aid of the polymerizing enzyme. The particle may effect a change in the electrical current detected by the plurality of nanoelectrodes (e.g., a redox cycling current modifying particle). The change in the electrical current detected may be used to identify the nucleotide binding to the nucleic acid strand, thereby identifying at least a portion of a sequence of the nucleic acid molecule. The polymerizing enzyme can be a deoxyribonucleic acid (DNA) polymerase, a reverse transcriptase, an RNA-dependent RNA polymerase or an RNA polymerase. The polymerizing enzyme can be DNA polymerase (DNAP) I, DNAP a, DNAP (3, AMV DNAP, T4 polymerase, T7 polymerase, RB69 polymerase, Bst polymerase, Dpo4 polymerase. The polymerizing enzyme may comprise one or more mutations. The mutation may confer increased stability (e.g., stability in the presence of a particular redox moiety). The mutation may increase binding affinity for non-natural (e.g., tethered) nucleoside triphosphate molecules. The mutation may provide a reactive moiety through which the polymerizing enzyme can be tethered to a surface. The mutation may alter the kinetics of the polymerizing enzyme (e.g., nucleoside triphosphate binding or nucleotide incorporation rate). The mutation may alter the exonuclease activity of the polymerizing enzyme.

One or more nucleotides coupled to a particle (e.g., a redox cycling current modifying particle) can be brought in contact with the nucleic acid molecule to be incorporated into the nucleic acid strand. The nucleotides can be of the same or different base types. The particles can have substantially identical or different sizes. In some cases, the particles have substantially identical sizes when a size difference of the particles is less than or equal to about 20%, 19%, 18%, 17%, 16%, 15%, 14%, 13%, 12%, 11%, 10%, 9%, 8%, 7%, 6%, 5%, 4%, 3%, 2%, 1% or less. As an example, two particles are considered having substantially identical sizes when the particles have diameters of 55 nm and 50 nm respectively, and a size difference is about 10%. In some cases, the particles have different sizes when a size difference is greater than or equal to about 30%, 35%, 40%, 45%, 50%, 55%, 60%, 65%, 70%, 75%, 80%, 85%, 90%, 95%, 100%, 150%, 200%, or more. As an example, two particles with respective diameters of 100 nm and 200 nm have a size difference of about 100%, thus are considered having different sizes. When different types of nucleotides or bases (e.g., A, C, G, T, U) or modifications thereof are introduced into the solution, they can be introduced simultaneously, sequentially, or a combination thereof. Each type of nucleotides may comprise a particle. Particles coupled to different types of nucleotides may be the same or substantially identical, or may differ in one or more characteristics or properties. For example, the particles may differ in shape, material, size, porosity, density, or combinations thereof. Different particles may modify the redox cycling to different degrees, resulting in different electric current profiles, which can enable identification of different bases. Thus, when each type of the nucleotides (or bases) or modifications thereof is coupled to a different particle, the particles may each cause a signal change that is distinguishable from one another, which signal may be indicative of a presence or absence of a specific type of nucleotides or modifications thereof. In some cases, at least two types of nucleotides comprise the same type of particle. In some cases, a particle may have a plurality of nucleotides coupled thereto. When multiple base types use the same particle, the base type can be determined by only delivering one base type at a time. Thus the presence of the particle may indicate which base type is used and consequently determine this part of the sequence. In some cases, it may be desirable to add non-incorporable nucleotides to reduce the chance that a non-complementary base is incorporated.

Nucleotides of four different base types can be attached to different sized particles to distinguish between the four different bases. Having four different particle types can allow all four bases to be delivered at the same time which can improve throughput. If there are two different particles this may allow two bases to be delivered at the same time.

The redox cycling may occur in a recess in a substrate (e.g. well). The redox cycling may occur in a nanopore. The solution comprising a reversible redox moiety can be contained in a well. The well can comprise one or more nanoelectrodes of the plurality of nanoelectrodes. The plurality of nanoelectrodes may comprise an additional nanoelectrode external to the well. A molecule or a nucleic acid molecule can be immobilized to a support in the well. The polymerizing enzyme can be immobilized in the well. The support can be a bead or a surface of the well. As an alternative, the redox cycling can be performed on a planar array. In some cases, the molecule or a nucleic acid molecule can be immobilized on the electrodes or to the surface between the electrodes.

The particle can modify the rate of redox cycling in a gap (e.g., a nanogap), and in such cases may be referred to as a redox cycling current modifying particle. The particle (e.g., a redox cycling current modifying particle) can include coatings to reduce non-specific binding, increase the effective diameter, reduce agglomeration, improve wetting or include binding compounds such as tethered nucleotides.

The particle can comprise oxides such as silicon dioxide. The particle can comprise a polymeric or metallic material. The polymeric material can be selected from a group including, but not limited to, polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), and a derivative thereof.

The biomolecule or nucleotide can be coupled to the particle by a tether. The tether can be coupled to the nucleotide by a phosphate group of the nucleotide such that the particle can be decoupled from the nucleotide when the nucleotide is incorporated into the nucleic acid strand. For example, the tether can be coupled to the terminal phosphate of a nucleoside triphosphate, such that the tether remains attached to a pyrophosphate cleaved from the nucleoside triphosphate upon nucleotide incorporation. The tether can comprise a polymeric material. The polymeric material may be branched, unbranched, partially branched, cross-linked, non-cross-linked, or partially cross-linked. The polymeric material may be hydrophilic. The polymeric material can be selected from a group including, but not limited to, polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof.

A particle can comprise a plurality of polymers on a surface (e.g., an exterior surface) at a density that can reduce non-specific binding to the particle. The polymer can be selected from a group including, but not limited to, polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof.

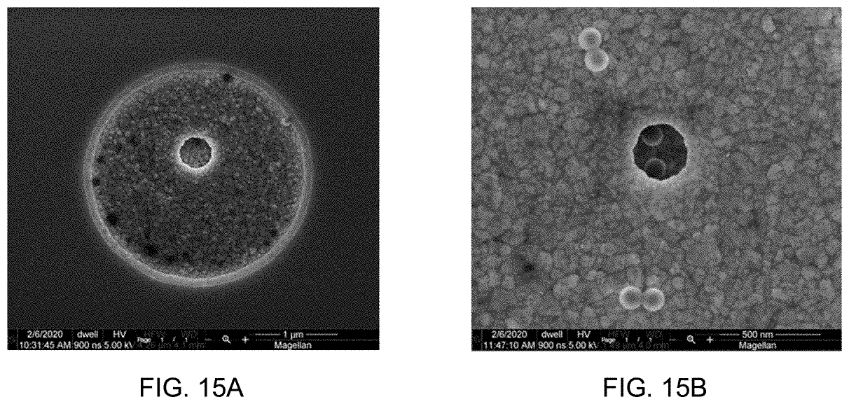

FIG. 15B shows an SEM image of a portion of the sensor. This image shows potential issues that may be associated with inadequate control of non-specific binding (e.g., when two beads bound in well and two beads stuck together on top electrode).

One or more nucleotides can be coupled to the particles by tethers. The nucleotides can be the same base type, and the tethers can have substantially similar sizes. The nucleotides can be different base types, and the tethers can have different sizes. The tethers of different lengths can have different release profiles of the nucleotides such that they result in different electric current profiles, which enables identification of all the different bases.

The particle can have one or more charges that electrostatically attract the reversible redox moieties toward the particle. A plurality of particles can comprise a plurality of polymers on their surfaces at different densities. The plurality of polymers can passivate the particles and reduce non-specific binding, which may prevent the particles from binding to other surfaces. Polymers disposed on a particle surface can slow down the binding rate of the particles. The polymers can further increase the effective diameter of the particle, reduce agglomeration, and improve wetting. Polymer density can be varied to change the modifying effect of a particle to redox cycling (e.g., may alter the degree to which a particle modifies a redox cycling current between two electrodes.

A lack of a change in the electrical current detected with the use of a particle coupled to a nucleotide can be indicative of an abasic site and can be used to identify an abasic site on a nucleic acid strand.

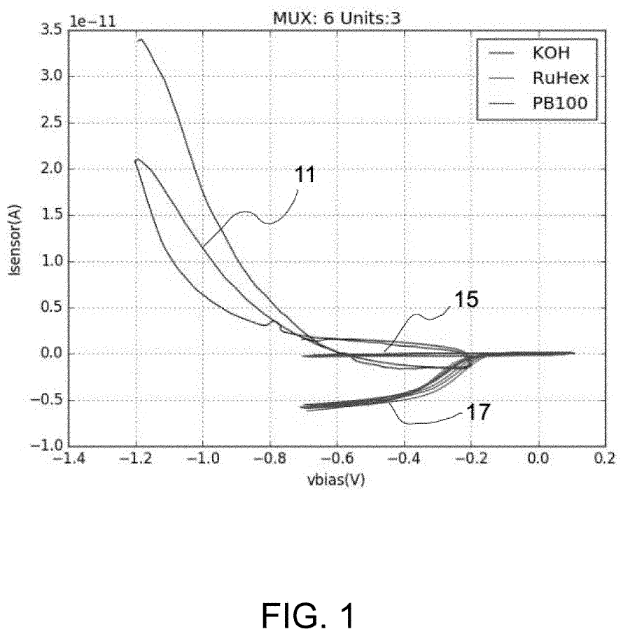

FIG. 1 shows an experimental result of a redox cycling done on a sensor chip comprising nanoelectrodes using 1 mM of Hexaammineruthenium (III) chloride in 100 mM phosphate buffer. FIG. 1 illustrates the measurable electrical current difference from a KOH voltage sweep cleaning 11, a voltage sweep of 100 mM phosphate buffer 15, and a voltage sweep using 1 mM Hexaammineruthenium (III) chloride in 100 mM phosphate buffer 17.

Redox cycling can create a measurable electrical current with even small sensors, such as nanosensor comprising nanoelectrodes. The small cross-section of the nano-gap can facilitate modification of redox cycling electrical current with the use of particles.

The sensors can be used in applications where binding is important. The sensors can be used in generic binding assay. The sensors can be used as a sensitive detector for biomacromolecules such as enzymes, antibodies, and DNA. The use of reversible redox moieties and sensors that facilitate redox cycling can dramatically increase the signal level.

The sensor can be configured to detect individual or multiple biomacromolecules. A nanoelectrode pair on a larger scale can be used to detect the presence of multiple biomacromolecules. The nanoelectrode pair may be modified to attach a specific binding moiety such as an antibody. A calibration sample may be used to calibrate the nanoelectrode pairs.

The electrodes disclosed herein may be used to distinguish between different molecules. The signal change from an individual molecule may be used to identify a specific substance in a mixture allowing the concentration of multiple biomacromolecules to be identified.

Systems described herein may be used in the detection or identification of a biomolecule. The detection system can be used to detect a single particle. The detection system may be used for the detection of single-molecule binding events and can be incorporated into a massively parallel sensor chip with millions of nanosensors. The system may use reversible redox compounds in solution that can diffuse between two closely spaced nanoelectrodes (nanoelectrode pairs). When appropriate voltages are applied to the nanoelectrodes, the reversible redox compound can be oxidized on one electrode, diffuse to the other electrode and be reduced. This process can transfer one or more electrons per cycle. There typically are many reversible redox molecules in the nanogap and each one can make thousands of trips between the nanoelectrodes each second, generating a measurable redox cycling current.

If a redox cycling current modifying moiety, such as a nanoparticle (or a macromolecule such as protein), comes into the nanogap, it may alter the redox cycling efficiency to produce a measurable change in the redox cycling electrical current. This can occur as an electrical current decrease or an electrical current increase. A current decrease may occur if the modifying moiety impedes the diffusion between the nanoelectrode pairs or occludes the electrode surface causing a decrease in redox cycling efficiency. An electrical current increase may occur if the presence of the modifying moiety between the nanoelectrodes for example, by increasing the local concentration of redox compounds.

One application of such a sensor chip can be to determine the sequence of nucleic acid polymers such as DNA and RNA using the sequencing by synthesis approach. Because multiple nanosensors (e.g., each nanosensor) on the sensor chip can detect an individual binding event, single-molecule sequencing by synthesis can be performed without the need for expensive and problematic clonal amplification. Single-molecule systems can avoid dephasing problems, which can enable longer read lengths. Most of the current single-molecule sequencing systems use optical detection of fluorescent moieties, which requires expensive laser and camera subsystems, and may be severely limited in bandwidth (due to camera speeds) and throughput (due to the diffraction limit of light requiring low sensor density). The higher sample preparation cost and additional hardware cost make DNA/RNA sequencing more expensive. Compared to optical detection, electrical detection offers the potential for ultrahigh sampling rates and sensor density, yielding dramatically improved parallelism and throughput. The present disclosure enables a chip, or a set of chips, each comprising a plurality of sensors to sequence DNA. A chip may comprise at least 100, at least 500, at least 1,000, at least 5,000, at least 10,000, at least 50,000, at least 100,000, at least 500,000, at least 1,000,000, at least 5,000,000, at least 10,000,000, at least 50,000,000, at least 100,000,000, at least 500,000,000 sensors, or more. The sensor chip may be cleaned and reused multiple times enabling even lower sequencing costs.

In addition to DNA sequencing, the sensor chip/approach can be used to study single-molecule protein-protein and protein-DNA binding and interaction kinetics, including association and dissociation constants. Moreover, the technique can be adapted for ultra-sensitive protein detection using the nanoelectrode pairs.

The sensor comprising the electrodes can be integrated into a semiconductor chip to enable readout from many sensors. In some cases, the sensors can be read by circuitry that measures the electrical current from at least one of the nanoelectrodes. In some cases, this readout can be software configurable. In some cases, the readout can address a subset of all sensors. In some cases, more than 100 million sensors can be built onto a silicon chip to facilitate massively parallel detection. In some cases, the more than 100 million, 200 million, 300 million, 400 million, 500 million, 600 million, 700 million, 800 million, 900 million, 1 billion, or more sensors can be built onto a silicon chip.

The chip may include other functionality such as temperature measurement. In some cases, the chip may include potentiostat circuitry to control or monitor the voltage of the fluid in contact with the chip. In some cases, the system may include potentiostat circuitry off the chip to control or monitor the voltage of the fluid in contact with the chip.

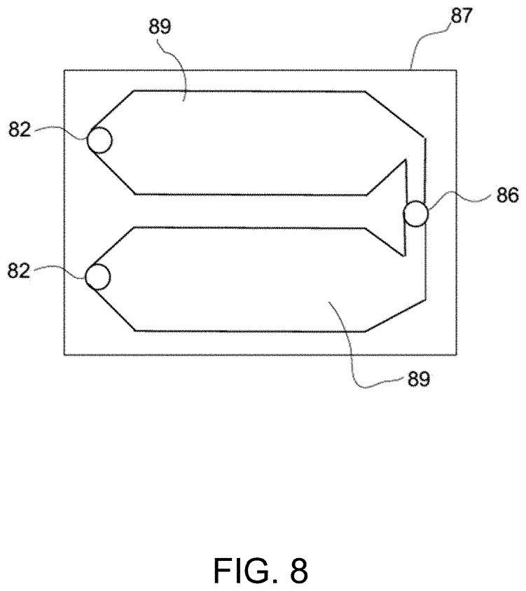

The sensor chip may be configured with one or more flow channels. Flow channels may include one or more inlets and outlets. In some cases, separate fluid channels can be combined into fewer inlets or outlets. In some cases, a sensor channel may have a narrow depth (<500 um, 250 um 100 um, 50 um, 25 um, or less) above the sensors. FIG. 8 shows a schematic of a possible fluidic interface 87 of a sensor chip. In this example, two inlets 82 are used which flow into channel areas 87 and then are combined into a single outlet 86. In some cases, the fluidic channels may have pillars (e.g., greater than or equal to about 2, 3, 4, 5, 6, 7, 8, 9, 10, 15, 20, 25, 30, 35, 40, 45, 50 pillars, or more) to control the gap spacing. In some cases, the fluidic channel may be compressed to ensure the depth is controlled. In some cases, an adhesive or other bonding approaches can be used to attach the flow channels assembly to the chip and create a seal. In some cases, the channel depth may vary. In some cases, the fluidic channel may be created by a transparent material such as plastics or glass.

In some cases, the sensor chip can be configured to enable temperature control of the sensor chip. In some cases, the temperature may be controlled by a heater or Peltier module associated with the chip. In some cases, the chip may facilitate temperature control by an external heater or Peltier module.

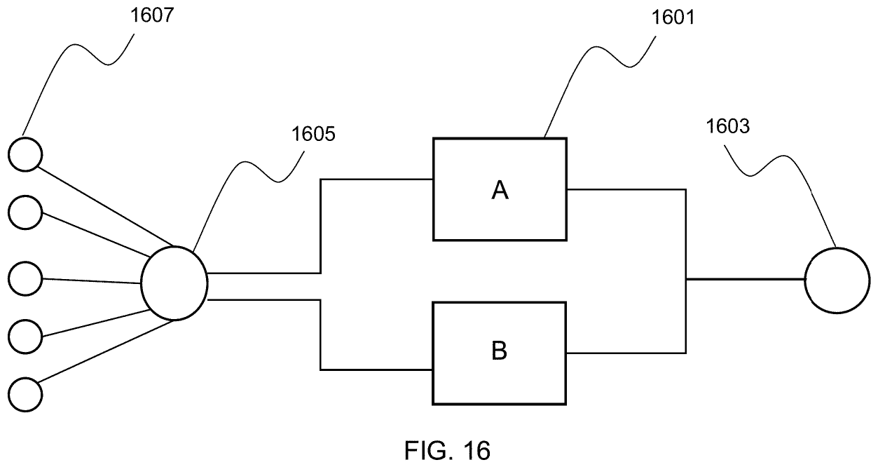

Multiple chips may be used in an instrument. FIG. 16 shows a schematic of a simple system with two chips. Multiple reagents 1607 may be connected to a valving system 1605 that can direct the fluid into two chips 1601 designated A and B. In some cases, a chip may comprise an independent temperature control system and an independent reference/counter electrode system. The outflows from a chip may be combined to deliver to a waste reservoir 1603. In some cases the operation of one or more chips may be coordinated. For example, one chip may have fluid delivery while another chip may have the sensors read out.

The particle can repeatedly modify redox cycling levels. The particle may be a large molecule such as a protein. The particle may be a solid particle such as silica, polymers such as polystyrene, PMMA, etc, gold or other metals, or metallic compounds such as iron oxide. In some cases, the particle may be a composite material such as a coated magnetic particle. In some cases, the particle may be porous. In some cases, the particle may be a microsphere or a nanosphere.

FIG. 13 shows data from a blocking experiment wherein 100 nm polystyrene nanospheres diffuse freely into a 200 nm diameter sensor with 10 mM ferrocene dimethanol in a 100 mM phosphate buffer and a voltage difference of 0.6V. This data was filtered with a 45 Hz longpass filter. The downward pulses are the result of the electrical current output being reduced by the presence of particles.

For nucleic acid sequencing by synthesis methods, four color fluorescent sequencing is often used. Multiple signals can also be generated using particles (e.g., redox cycling current modifying particles). In some cases, particle size can be varied to change the amount of redox cycling electrical current. In some cases, a plurality of particles may have a tight size distribution (monodisperse) in order to generate consistent signals. Two or more differently sized particles can be used, each optionally comprising a different binding compound attached. For nucleic acid sequencing, the binding compound may be one of the bases (for example, G, A, C or T for DNA).

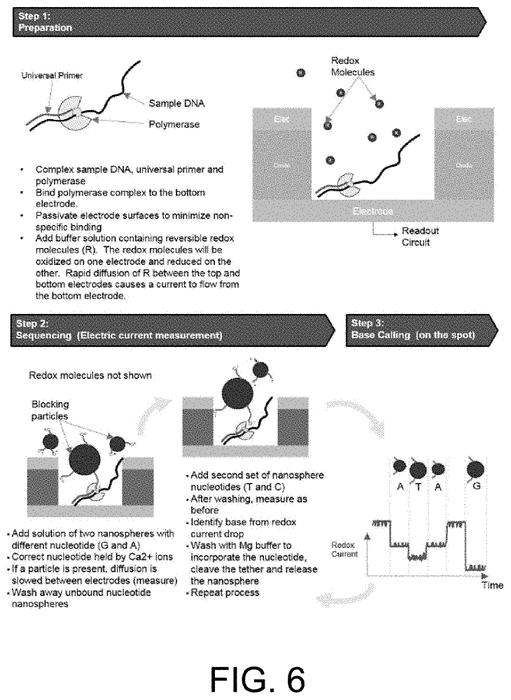

FIG. 6 schematically illustrates a method of DNA sequencing using different size particles. A system may comprise a complex biological sample (e.g., a sample comprising multiple cellular components, proteins, lipids, small molecules, and nucleic acids). The system may also comprise reagents for nucleic acid synthesis, such as a primer (e.g., a universal primer), nucleotides (e.g., nucleoside triphosphates), and a polymerase. The polymerase may be immobilized adjacent to an electrode (e.g., near an outer edge of the electrode or on the electrode). The electrode may be passivated, for example to minimize non-specific binding to the electrode. The electrode may be an electrode of an electrode pair. The system may comprise a buffer comprising reversible redox molecules. The reversible redox molecules may be oxidized on a first electrode and reduced on a second electrode (e.g., the first and second electrodes of an electrode pair, respectively). Diffusion of the reversible redox molecules between the electrodes of the electrode pair may generate a current between the electrodes of the electrode pair.

The nucleotides may be coupled to particles. Different nucleotides may be coupled to particles of different sizes. For example, a first particle comprising a first size may be coupled to a first nucleotide (e.g., adenine) or a first set of nucleotides (e.g., adenine and guanine), and a second particle comprising a second size may be coupled to a second nucleotide (e.g., cytosine) or a second set of nucleotides (e.g., cytosine and thymine). A nucleotide coupled to a particle may bind to a polymerase, which may immobilize the particle adjacent to (e.g., between) a gap between the electrode pair. If a particle is disposed between the electrode pair, diffusion of the redox molecules between electrodes may be inhibited, which may decrease the current between the electrodes of the electrode pair. Different particles may decrease the current by different amounts, thereby enabling identification of the nucleotide or set of nucleotides coupled to them.

Different particles may be added to the system at different times. In some cases, a first particle may be added to a system at a first time, and removed (e.g., through washing) prior to the addition of a second particle. Addition of a cation (e.g., a divalent cation such as Mg²⁺) may activate a polymerase to decouple a nucleotide from a particle (e.g., cleave a tether coupling the nucleotide to the particle), which may release a particle from a polymerase, and further may result in diffusion of the particle away from the electrode pair.

FIG. 14 shows a histogram with Gaussian fit of current blocking current levels from three runs with different diameter polystyrene nanospheres diffusing into 200 nm diameter sensor wells. This standard deviation from these measurements may be caused by a number of factors. First, a freely diffusing bead may not reach the bottom of a well, and consequently may partially rather than fully block redox cycling. Second, when a polymerase is present, a bead may be more likely to consistently bind in the same spot, as the polymerase may bind to a biomolecule attached to the bead. For the data in FIG. 14, the bead can go anywhere in the sensor and modeling has shown that the amount of redox cycling blocked depends upon the position. Third, the data in FIG. 14 was generated from multiple sensors. In some cases, variations in sensor diameters can also change the amount of redox cycling blocked by a bead.

In some cases, one type of particle can be separately coupled to more than one nucleotide (e.g., a first subset of a plurality of particles of a single type are coupled to a first type of nucleotide and a second subset of the plurality of particles of the single type are coupled to a second type of nucleotide). For example, nucleotides A and G can be labeled with large particles and nucleotides C and T with small particles. If A and C are delivered together, the binding nucleotide can be determined by the redox cycling current change. Later, G and T can be delivered to allow the detection of these bases.

In some cases, a lack of a particle can be used to determine the binding of a particular nucleotide or other binding moiety. For example, a large, medium, small and zero redox cycling current change can be created by using nucleotides A, G, C, T coupled to four different particles, allowing identification of all 4 bases in one measurement.

Sometimes it can be desirable to identify whether the base has no complement as in the case of abasic site. A lack of redox cycling current change can be used to identify a modified base which doesn't have a complement.

It may be desirable that the particle may be able to diffuse freely and not stick to any other surface other than the binding target. A particle may be passivated with molecules such as polyethylene glycol (PEG) or alkane polymers, or other compounds to reduce non-specific binding. Such passivation can be done by covalently or non-covalently bound molecules.

It may be desirable to add compounds that stick to surfaces in order to reduce non-specific binding. Examples include surfactants such as Tween® 20, Tween® 80, Triton™ X-100, and protein blockers such as Bovine Serum Albumin (BSA).

In some cases, the binding moiety, such as a nucleotide, along with the tether may be adequate to prevent non-specific binding. For example, a particle may be coated with nucleotides on PEG tethers. In some cases, the tether may be longer than the coating to minimize non-specific binding. For example, the tether size may be 1 k, 2 k, 5 k, 10 k, 20 k Daltons or more than the coating for non-specific binding. In some cases, the tether may be attached to the coating to prevent non-specific binding. In some cases, the tether may be directly attached to the bead. In some cases, the tether may be sufficiently short to confine a particle within an electrode gap upon binding of a tethered biomolecule to a protein. The nucleotides can allow binding and detection, but the long PEG tether may prevent the particle from binding to surfaces other than the intended binding spot (a polymerase with a complementary base).

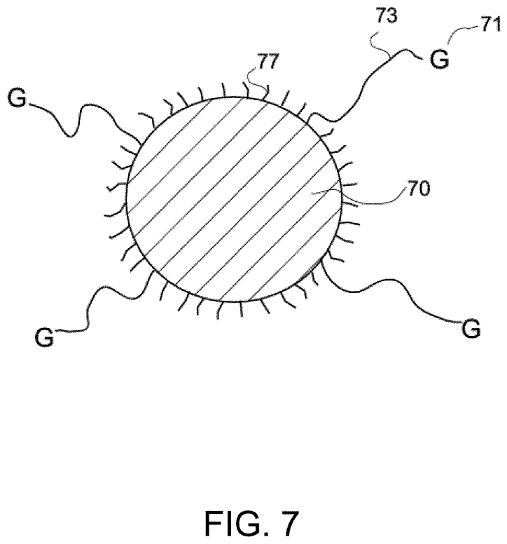

A particle may comprise (e.g., be coupled to) multiple binding moieties. A particle may comprise more than 10, more than 40, more than 100, more than 200, more than 300, more than 400, more than 600, more than 800, more than 1000, more than 2000, more than 3000, more than 4000, more than 10000, more than 40000, or more binding moieties. A higher surface loading of binding moieties may facilitate a lower concentration of particles. FIG. 7 shows a schematic of a particle 70 with a coating 77 to prevent non-specific binding. The coating to prevent non-specific binding may use short polymers packed tightly that stick out from the surface creating a “bottle brush” coating. Alternately, longer polymers may be used or a mixture of long and short. A nucleotide 71, shown as G, is attached to the particle 70 using a tether 73. This tether may be connected to the base through the phosphate chain such that incorporation of the nucleotide into the nucleic acid chain releases the particle 70. In some cases, the phosphate chain may have 3, 4, 5, 6 or more phosphate atoms, for example, to improve the binding and incorporation kinetics.

In some cases, the density of the binding moieties may be varied. For example, a lower density may cause a slower binding rate, and this may be used to help determine the binding moiety. For example, two different sized particles with two levels of surface densities can be used. This can allow identification of 4 different binding moieties, which can be useful for the 4 nucleotide bases (A, G, C, and T for DNA).

Reversible redox molecules may be selected to minimize inhibition of the polymerase enzymes. Reversible redox molecules may include the following: Hexaammineruthenium(III) chloride, methyl viologen dichloride hydrate, hexacyanoferrate(II)trihydrate, ferricyanide, 4-aminophenyl or ferrocene compounds such as ferrocenedimethanol, 1,1′-Ferrocenedicarboxylic acid or (Ferrocenylmethyl)trimethylammonium Chloride, or methylene blue. In some cases, the concentration of the reversible redox molecule may be less than 100 millimolar (mM), 30 mM, 10 mM, 3 mM, 1 mM, 0.3 mM or 0.1 mM.

If a nucleic acid base complement is not available in the solution a polymerase may incorporate the wrong nucleotide. In some cases, there may not be enough unique particles for all the binding moieties being tested. For example, there may be two types of particles, large and small. For DNA sequencing, the two types of particles can be used for the two nucleotides A and G in one test cycle and C and T in another. To avoid incorrect binding, an unincorporable nucleotide may be available in solution. In some cases, the unincorporable nucleotides can be a nucleotide with substitutions in the phosphate chain such as replacing the alpha, beta or both phosphates with an arsenic, Sn, Bi or N, a PNA nucleotide, an L-DNA nucleotide, a locked DNA nucleotide, a ribonucleotide, an adenine monophosphate, an adenine diphosphate, an adenosine, a deoxyadenosine, a guanine monophosphate, a guanine diphosphate guanosine, a deoxyguanosine, a thymine monophosphate, a thymine diphosphate 5-Methyluridine, a thymidine, a cytosine monophosphate, a cytosine diphosphate cytidine, a deoxycytidine, a uracil monophosphate, a uracil diphosphate, a uridine, or a deoxyuridine.

One way to modify the effect of a particle can be to change its nominal position in the sensor. In some cases, identical particles may be tethered to different nucleotides (A/G/C/T) by tethers of different lengths, resulting in different current signatures, which enables identification of all four bases.

In some cases, the particle may have a charge. The charge may directly or indirectly attract redox-active compounds, which may result in a higher local redox compound concentration. By attracting a higher local redox concentration, the redox cycling current may increase. In some cases, the particle may be porous which can increase the surface area allowing more redox molecules to be attracted. In some cases, the particle may have a corrugated surface, or have a spiky surface, or be entirely made of thin rods connected at the center of the particle, etc. to attract a higher concentration of redox molecules into the nanogap. In some cases, the particle may be coupled to charged molecules, such as DNA, in order to increase the local redox compound concentration.

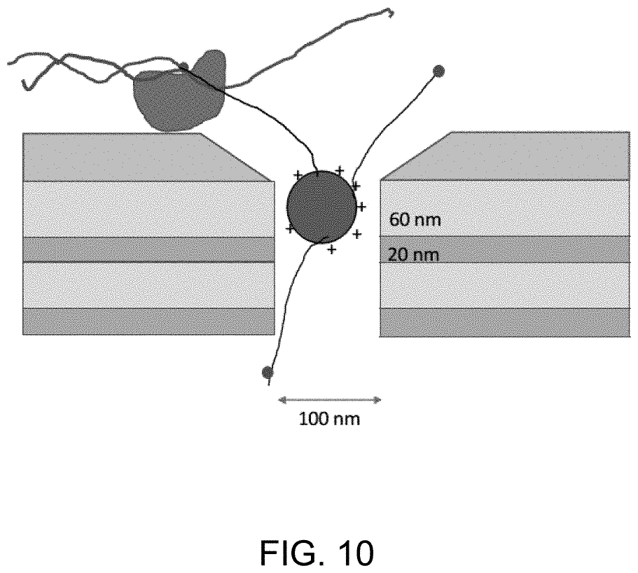

A mixture of charged and uncharged particles may be used to allow multiple signal levels. Multiple signal levels may be associated with different binding moieties such that all four bases of the nucleotides can be identified simultaneously. Modified redox cycling may also be useful in nanopores. FIG. 10 shows a nanoelectrode pair used with a synthetic nanopore with a diameter of 100 nm. In some cases, the polymerase may be held on one surface near the nanopore. In some cases, the particle may be positively charged so that it can be pulled into the nanopore while the negatively charged DNA is not.

In some cases, the nanopore cross section area can be less than lum², 800000 nm², 600000 nm², 400000 nm², 200000 nm², 100000 nm², 80000 nm², 60000 nm², 40000 nm², 30000 nm², 15000 nm², 10000 nm², 8000 nm², 6000 nm², 4000 nm², 3000 nm², 1000 nm², 300 nm², or 100 nm².

In some cases, the gap between the two electrodes of the nanoelectrode pair can be less than 800 nm, 400 nm, 200 nm, 100 nm, 50 nm, 25 nm, 10 nm.

The sensor may be cleaned between detection assays. Cleaning can include acid, base, detergent or solvent washing. Electrochemical cleaning may be used such as with H₂SO₄ or KOH voltage cycling.

A surface chemistry protocol may be applied to sensor chips comprising electrodes before use. In some cases, the surface chemistry protocol may be preceded by a cleaning protocol. The cleaning protocol may include washing by solvents, acids, bases, etc. The electrodes may be cleaned by voltage cycling of the electrodes relative to the reference voltage of the solution. The surfaces may be cleaned by plasma treatment.

In some cases, a cleaning protocol can be used to dissolve any beads that may have bound to the sensor chip. In some cases, the chip may be reused multiple times to decrease the consumables cost. In some cases, the instrument may monitor how many times the chip has been used and not allow chips that have already been used to continue to operate.

After cleaning, it may be desirable to add surface treatments. The sensor chip surfaces can be passivated. The oxide or plastic surfaces can be treated to minimize non-specific binding and enhance wetting. The electrode surfaces may similarly be passivated. In some cases, thiols are used to attach passivation molecules to the electrodes. In some cases, different coatings may be used on different electrodes. For example, in some cases the bottom electrode in a well may have coatings that allow binding of DNA or the polymerase. In some cases, differential electrode coatings may be facilitated by using different voltages during the coating formation. The electrode surface may be passivated with an optimized density of PEG or alkane polymers such as to minimize non-specific binding while still preserving access to the electrode surface for the diffusing redox cycling moieties.

Some of the sensor surfaces may be modified to enable attachment of a polymerase. The attachment may target the nano-gap area. Attachment methods can include adding covalent linkers as well as non-covalent linkers such as biotin-avidin. The nanogap surfaces may be modified to allow the attachment of the target nucleic acid. The linker to the nanoelectrodes may be attached by thiol groups. A target capture oligo or a part hybridized to it, such as a universal primer, rather than the polymerase may be bound to the electrode. The binding can include a thiol attachment. The binding to the electrode may be due to a nucleic acid hybridization. The attachment procedure may be done to maximize the number of sensors that have a single bound polymerase or single bound nucleic acid. This may be accomplished using an appropriate dilution of the polymerase or nucleic acid to control binding density. In some cases, the polymerase may be bound to the target nucleic acid before delivery to the nanogap.

### (A) ELECTRODE FABRICATION AND CHIP DESIGN

Electrode pairs can be fabricated using various approaches, such as photolithography. Electrodes of the electrode pair may be nanoelectrodes. The electrodes can be insulated except for a small fluidic exposed area. In some cases, the electrodes of the nanoelectrode pair can be coplanar (e.g., horizontally disposed relative to a surface of the substrate). In some cases, the nanoelectrode pair may be created by removing material from a previously connected electrode. This removal can be done by reactive ion etching, focused ion beam milling or other fabrication methods.

The electrode material may include corrosion resistant metals such as: Au, Ag, Pt, Pd, Rh, Ru, Os, Ir, Ti, Nb, or Ta or alloys or coating using these metals. In some cases, adhesion layers between the metal and oxides may be used. In some cases, the adhesion layer may be chromium or titanium. In some cases, the electrode material may be created by the conversion of a poor conductor into a conductive material. For example, polysilicon may be converted to platinum silicide.

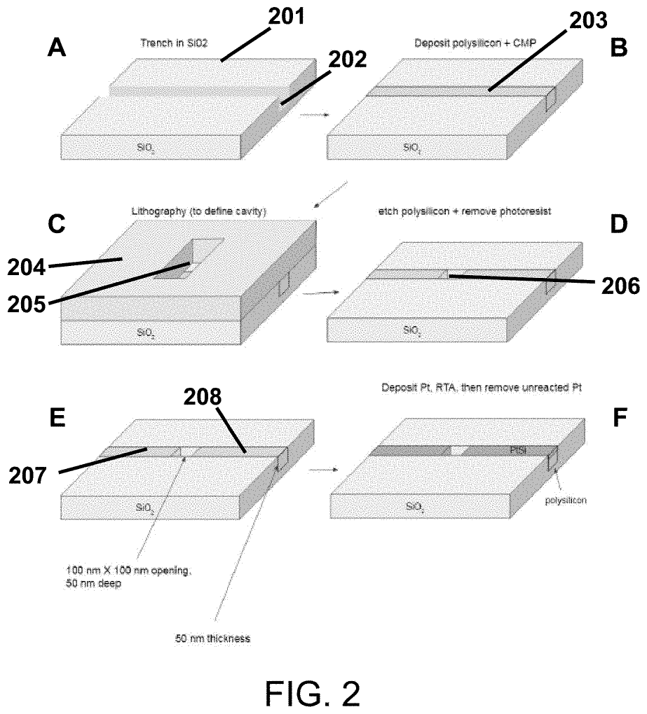

(a) FIG. 2 shows a process flow for creating a coplanar electrode pair using platinum silicide. As shown in panel A, a trench 202 may be etched in a substrate 201, such as SiO₂. As shown in panel B, the trench may be filled by a silicide forming deposit 203, such as polysilicon. Optionally, the surfaces of the substrate and deposit may be planarized, for example by chemical-mechanical polishing, slurry polishing, or electrochemical mechanical planarization. Panel C depicts the deposition of a resist layer 204 over the substrate with an opening 205 above a portion of the silicide forming deposit. As is shown in panels D and E, the exposed silicide forming deposit may be etched, and the resist layer may then be removed, yielding a gap (e.g., a nanogap) between two silicide forming deposits 207 & 208. As depicted in Panel F, a layer of platinum, titanium or other appropriate metal may then be applied over and reacted with the silicide forming layers (e.g., by a rapid thermal annealing process) to form metal silicide electrodes from 207 & 208. Any remaining metal may then be removed. The resulting gap between the metal silicide electrodes may have a width comparable to a dimension of the opening 205 in the resist layer depicted in panel C, while the depth of the gap may depend on the depth of the trench 202 etched into the substrate 201. Thus, the fabrication process outlined in FIG. 2 provides an electrode pair fabrication method with control over gap dimensions. In some embodiments, carbon electrodes such as graphite or glassy carbon may be used.

The nanogap can be formed by using a sacrificial material/block, created prior to fabricating the nanoelectrodes. The electrode coated block may then be planarized, for example by chemical, mechanical polishing thus creating a nanoelectrode pair. In some cases, the remaining sacrificial material may be removed, for example by etching. This method may eliminate the need to etch the nanoelectrodes while creating the nanoelectrode pair.

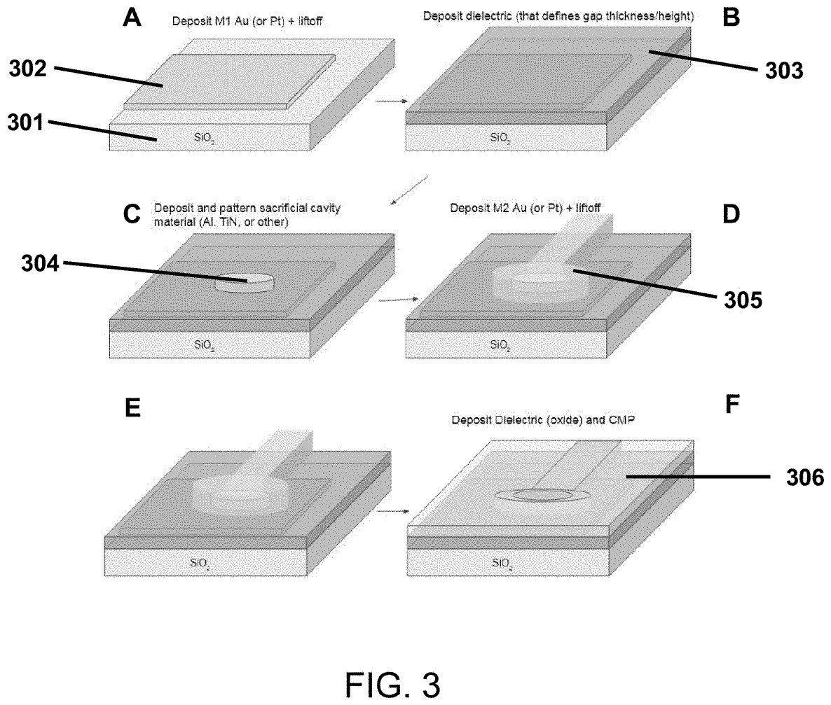

FIG. 3 shows the first part of a process for cavity creating a nanogap using a sacrificial material. In a first step depicted in panel A, a first metal layer 302 is deposited on a substrate 301 (e.g., SiO₂). The first metal layer may be gold, platinum, or any other suitable material. As shown in panel B, a first dielectric layer 303 may then be deposited over the first metal layer and substrate. Next, as is shown in panel C, a sacrificial material 304 is deposited on the dielectric layer at a position above the metal layer. A second metal layer 305 may then be deposited over the sacrificial layer, as is depicted in panels D & E. A second dielectric layer 306 may then be deposited (panel F), and the system may be planarized to a depth sufficient to expose the sacrificial material 304 through the second metal layer 305.

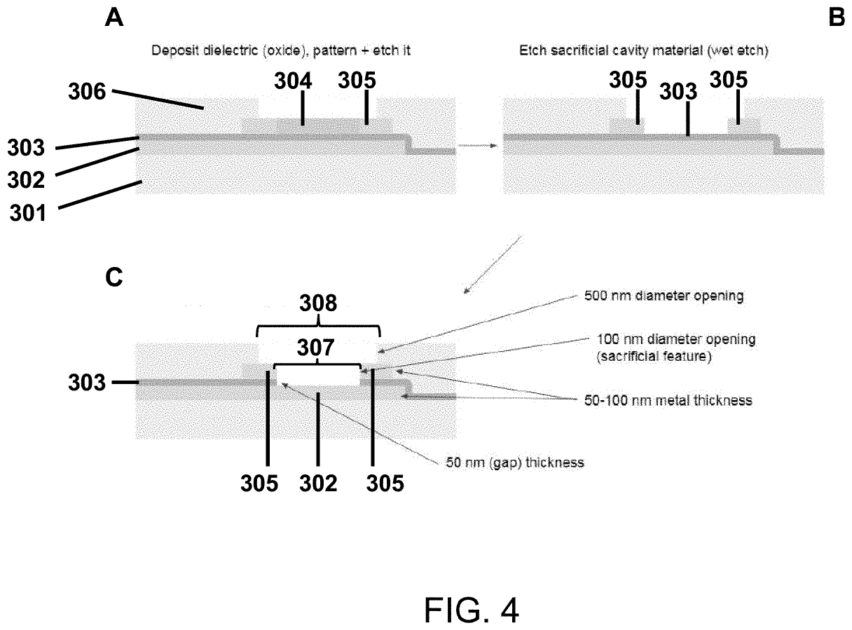

FIG. 4 shows the later parts of the process flow in cross-section, and contains the same labeling scheme as FIG. 3 and depicting the same structures. As is depicted in panel A, the second metal layer 305 and sacrificial layer 304 may be partially etched, thus creating a well above the sacrificial layer. This may be performed by any suitable method, including deposition of a patterned dielectric or resist layer followed by etching and removal. As is shown in panel B, the remaining portion of the sacrificial may be removed (for example by a wet etch), exposing the first dielectric layer 303. Further etching depicted in panel C may then be used to remove the exposed portion of the first dielectric layer to expose a portion of the first metal layer 302, and thus generating an electrode pair between the first 302 and second 305 metal layers, with a gap size equal to the thickness of the first dielectric layer 303. Thus, the electrode gap size may be selected and controlled for during fabrication. The fabrication process generates a cavity 307 within a well 308. The well may comprise a larger diameter than the cavity (e.g., 500 nm vs 100 nm). This difference may leave a portion of the upper electrode 305 exposed, thereby increasing its surface area.

A device may comprise a well. A well may be a depression in a surface, such as a surface of a substrate. A well may comprise an electrode, an electrode pair, or a plurality of electrodes or electrode pairs. As used herein, the terms ‘cavity’ and ‘well’ may be used interchangeably. In particular cases, the term ‘cavity’ may specifically denote a well that is disposed within a well (e.g., feature 307 in FIG. 4 panel C). A cavity may comprise an electrode pair. In some cases, a cavity comprises a first electrode of an electrode pair at a bottom end (e.g., the exposed surface of the first metal layer 302 in FIG. 4 panel C) and a second electrode of the electrode pair disposed closer to the top of the cavity (e.g., the second metal layer 305 in FIG. 4 panel C). The second electrode may comprise an exposed surface area facing the top of the well and disposed outside of the cavity. The second electrode may constitute a rim of the cavity. A cavity disposed within a well may comprise at most 2%, at most 3%, at most 4%, at most 5%, at most 8%, at most 10%, at most 12%, at most 15%, at most 20%, at most 25%, at most 30%, at most 40%, at most 50%, at most 60%, or at most 75% of the width of the well. A cavity disposed within a well may comprise a greater depth than the well. A cavity disposed within a well may comprise a shallower depth than the well. A cavity disposed within a well may comprise depth that is comparable to that of the well.

A device may comprise a plurality of cavities that share a common electrode. In some cases, the device comprises an upper electrode (e.g., analogous to the second metal layer 305 in FIG. 4 panel C) that is disposed within multiple cavities comprising separate lower electrodes (e.g., each analogous to the first metal layer 302 in FIG. 4 panel C). In some cases, the device comprises a lower electrode that is disposed within multiple cavities comprising separate upper electrodes. In some cases, one of the electrodes of the electrode pair may be common with one or more electrode pairs. This electrode may be used to set the voltage difference between the electrodes of the electrode pair. In some instances, this voltage is the same for all sensors, and the electrodes can be common. The other electrode, where the current is measured, can be separate.

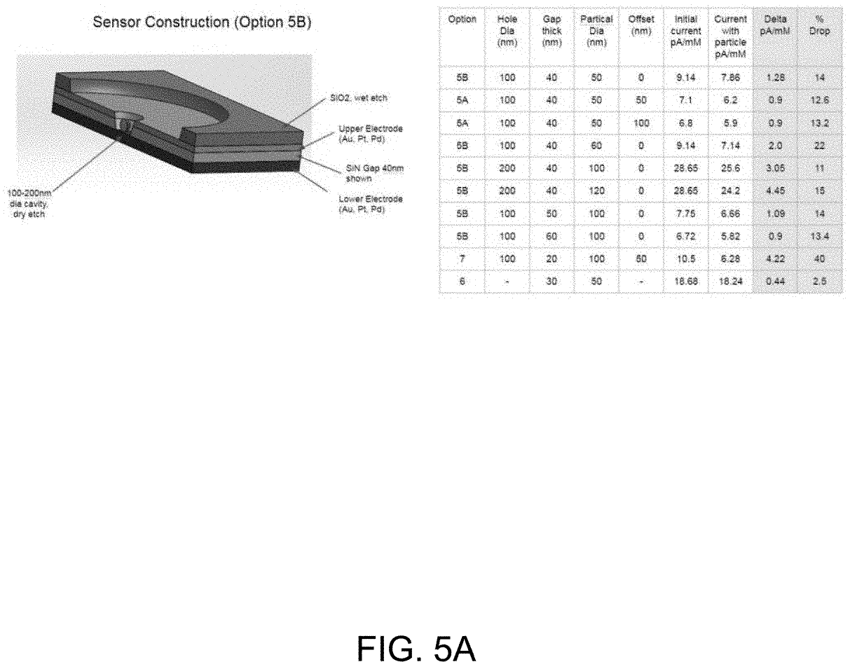

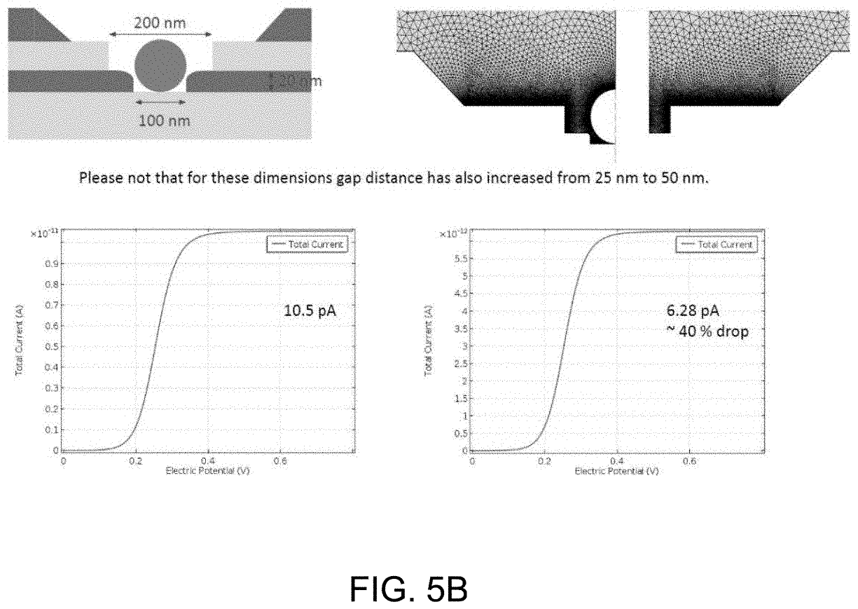

In other cases, the electrodes of the electrode pair (e.g., a nanoelectrode pair) can be on separate planes separated by an insulator. Accordingly, a first electrode of an electrode pair may be disposed above (vertically relative to) a second electrode of the electrode pair. The insulator can be one commonly used in semiconductor fabrication such as silicon dioxide, silicon nitride, tantalum oxide, aluminum oxide, hafnium oxide, etc. In some cases, the gap allowing fluidic contact between the nanoelectrode pairs can be created by reactive ion etching of a cavity that can allow fluidic contact between the electrodes. In some cases, the gap allowing fluidic contact may be formed by wet etching. In some embodiments, the top electrode partially encircles the cavity. In other options, the top electrode can fully encircle the cavity. If the top electrode fully encircles the cavity this can minimize alignment tolerance issues. FIG. 5A shows a cross section of a sensor concept where the nanoelectrode pair is contained in a well, is not coplanar, and includes a particle. COMSOL MULTIPHYSICS® (COMSOL) modeling results are shown in the table at the right of the figure, and provide estimated redox cycling current changes caused by particle intercalation into the well comprising the nanoelectrode pair in the presence of 1 mM redox cycling compounds.

In some cases, the fluidic exposed areas of one electrode can be larger (1.5×, 2×, 5×, 10×, or more) than the other electrode of the nanoelectrode pair. In some embodiments, the smaller electrode may be the electrode whose redox cycling current is measured. In some cases, the fluidic exposed area may be created by wet etching. In some cases, the cavity shape is cylindrical and may include a draft.

To facilitate redox cycling, the separation between the two electrodes of the nanoelectrode pair may be less than: 1000 nm, 800 nm, 400 nm, 200 nm, 100 nm, 50 nm, 25 nm, 10 nm, or less.

In some cases, the exposed surface area of the smaller electrode of the nanoelectrode pairs may be less than 10 um², 3 um², 1 um², 100000 nm², 30000 nm², 10000 nm², 3000 nm², or 1000 nm².

In some cases, the upper electrode may have a larger inner diameter than the exposed area of the lower electrode. For example, the top electrode inner diameter may be formed using the same mask as the cavity electrode (for example, to avoid alignment issues) and after wet etching the diameter may be larger. In some cases, the top electrode inner diameter may be more than 1.5, 2, 3, 4, 6, or more times the diameter of the exposed area of the bottom electrode.

FIG. 15A shows an SEM image of a sensor fabricated with a larger upper electrode. The area of the top level electrode is larger than necessary (3000 nm) due to equipment issues at the university fab.

The larger cavity created by having a larger inner diameter in the electrode than the dielectric may enable the use of larger nanospheres than practical with a deeper cavity. As shown in the modeling results from FIG. 5B, COMSOL modeling shows that the use of a larger nanosphere can result in a high level of current blocking. The traces shown are the current traces associated with a voltage sweep before and after adding a 100 nm diameter particle.

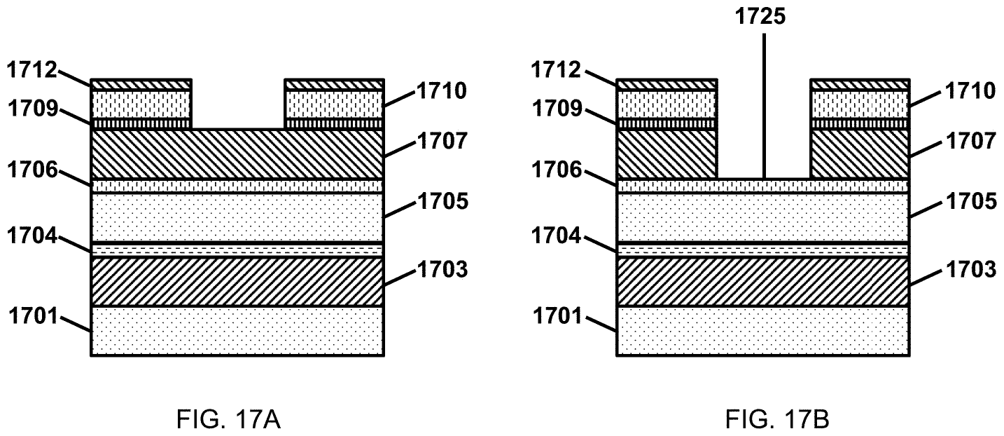

In some cases, a cavity in the top electrode may be created using liftoff. In some cases, the liftoff may use a double layer resist process. In some cases an adhesion layer such as Cr or Ti may be used to improve the adhesion of the electrode material to dielectrics. In some embodiments, an adhesion layer may be used as an etch stop for the cavity creation, for example, for reactive ion etching. In some cases an electrode may be used as a mask for etching the cavity. FIG. 17A shows a possible sensor cross section before the RIE of the insulator creating the well. An oxide layer 1703 may be created on a Si substrate 1701. Then a first electrode layer 1705 may be created with a lower adhesion layer 1705 and upper adhesion layer 1706 with boundaries defined by a liftoff process. An insulating layer 1707 may be created which may be SiO₂, SiN or another insulator. A second electrode 1709 may be added using a lithographic process and with boundaries and well top defined by a liftoff process. The second electrode layer may include a lower adhesion layer 1709 and upper adhesion layer 1712. FIG. 17B shows the sensor cross section after RIE. In some cases, the upper adhesion layer portion may be removed by the RIE process (imperfect etch stop) and some of the lower electrode may be etched. In some cases, any remaining adhesion layer may be removed from the exposed electrode surfaces by wet or dry etching. In some cases, piranha may be used to remove the exposed adhesion layer.

There may be a third electrode to control the voltage of the fluid in contact with the nanoelectrode pairs. In some cases, the third electrode may be upstream or downstream from the sensor chamber. In some cases, the third electrode may be integrated into the sensor chamber or be on-chip. In some embodiments, the third electrode may include a reference and may also include a counter electrode. In some embodiments, the surface area of the counter electrode may be larger than that of the combined sensor electrodes. In some cases, the surface area of the counter electrode may be increased by using a porous electrode.

In some cases, a chip may have a software selectable readout. For example, some sensors may have no polymerases or more than one. In some cases, data can be collected early in the run from a large percentage of the sensors to determine which sensors are not useful ((e.g., a sensor that has more than 1 or zero adjacently immobilized polymerases). By software selecting the sensors that are read these “not useful” sensors can be skipped, which can reduce readout time and decrease the amount of data to be collected.

In some cases, the systems may be designed to reuse chips. In some cases, cleaning protocols can be used to regenerate the electrode surfaces and remove any particles. In some cases, the system may prevent use of the chip after a number of runs. In some cases, the chip may include memory to facilitate counting the number of runs.

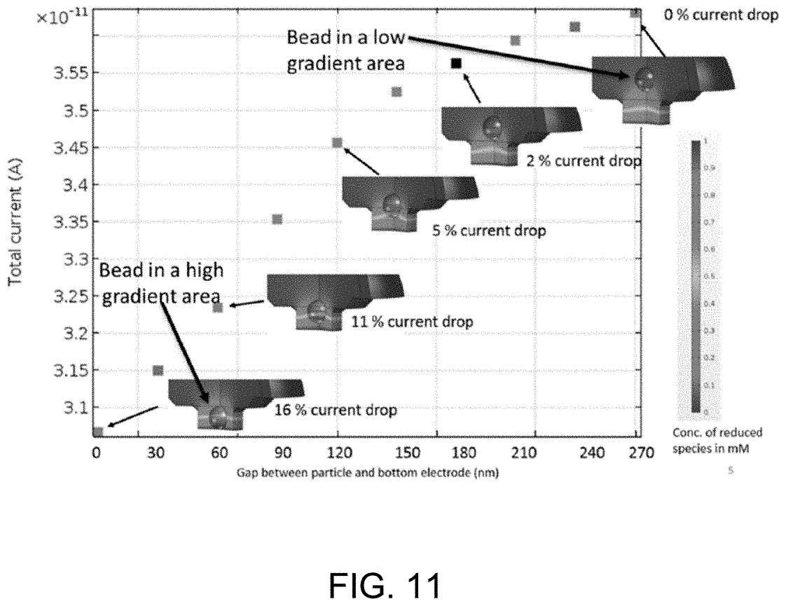

FIG. 11 illustrates a COMSOL modeling result of redox cycling with a nanoelectrode sensor. The modeling was done with 1 mM of ferrocene with a sensor cavity of 400 nm in diameter, a well depth of 200 nm, and a bead size of 200 nm. When the particle enters an area of high concentration gradient, the redox cycling blocking level improves. The gradient is largest where the color changes fastest. As can be seen in the figure, when the bead is near the bottom of the well, the gradient is fairly flat. This is because most of the redox molecules above the plane of the top electrode are reduced and only have to diffuse vertically to the bottom of the well to be oxidized. The current may comprise a proportionality to the ferrocene concentration. The drop was about 5 pA/mM when the particle was at the bottom.

### (B) NUCLEIC ACID SEQUENCING AND EPIGENETIC IDENTIFICATION

Detection of epigenetic information from modified bases may be desirable for DNA and RNA sequencing. Many DNA sequencing systems require bisulfate and other chemical modifications to identify methyl-cytosine. Since epigenetic information may be lost during amplification and not all the sequences in a sample may have the same modification, the ability to detect epigenetic modifications at a single molecule level can be desirable. It has been shown that binding and incorporation kinetics can be used to identify many modified bases. However, these events can be stochastic, so multiple measurements may be needed to determine the kinetics.

In some cases, a system can generate multiple measurements from the nucleotide binding to a single complementary base that may or may not be modified. In some cases, the nucleotide binding can be detected by the use of a particle. In some cases, multiple nucleotide binding events can be detected by using divalent cations that do not facilitate base incorporation. In some cases, multiple nucleotide binding events can be detected by using divalent cations that cause polymerases to have very slow base incorporation rates so that base incorporation is infrequent enough to be corrected by consensus analysis of the data, or so that a particle tethered to a nucleoside triphosphate remains bound to a polymerase for an extended period of time, thus enabling multiple measurements during a single polymerase binding event. For example, a divalent cation may cause a polymerase to perform nucleotide incorporation at a rate of at most 10 nucleotides per second, at most 1 nucleotide per second, at most 0.1 nucleotides per second, at most 0.01 nucleotides per second, at most 0.001 nucleotides per second. In some cases, divalent cations can include Ca²⁺, Mn²⁺, Zn²⁺, Fe²⁺, Be²⁺, Ba²⁺, Cd²⁺, Cr²⁺, Co²⁺, Ni²⁺, Cu²⁺, Pb²⁺ or Sr²⁺. In some cases, a divalent cation may be complexed to another species, such as ethylenediaminetetraacetic acid (EDTA) or citrate. In some cases, mixtures of divalent cations can be used. The polymerizing enzyme can be contacted with a first buffer comprising a first cation or a first mixture of cations to facilitate a transient binding of the nucleotide with the nucleic acid molecule; and the polymerizing enzyme can be subsequently contacted with a second buffer comprising a second cation or a second mixture of cations to facilitate incorporation of the nucleotide into the nucleic acid strand. In these conditions, particle diffusion away from the polymerase may be favored over incorporation of a second nucleotide from the bead, such multiple nucleotide incorporations by a single polymerase are minimized during contact with the second mixture. The first cation or the first mixture of cations can be selected from, but not limited to, Ca²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. The first cation or the first mixture of cations can comprise Ca²⁺, Zn²⁺, or a combination thereof. The second cation or the second mixture of cations can be selected from, but not limited to, Mg²⁺, Mn²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺. The second cation or the second mixture of cations can comprise Mg²⁺, Mn²⁺, or a combination thereof. The first cation can be Ca²⁺ and/or Zn²⁺ and the second cation can be Mg²⁺.

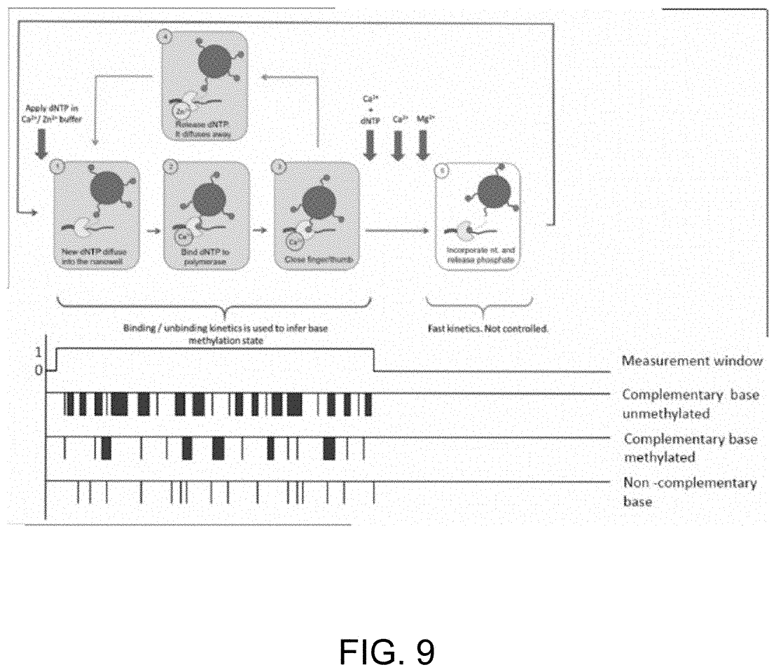

In some cases, mixtures of cations that include Ca²⁺ may be used to make the polymerase hold a nucleotide for a long time. A Ca²⁺ containing buffer can be used to hold the nucleotide for extending the measurement time or for later incorporation. FIG. 9 shows a schematic of a process to capture epigenetic information. A labeled nucleotide may be introduced along with a buffer which may contain a mixture of cations. When some divalent cations are associated with the polymerase, the polymerase may hold onto the nucleotide for a longer time. Some divalent cations may allow the polymerase to release or incorporate a bound nucleotide. Certain mixtures of cations may allow binding and release of labeled nucleotides. The appropriate cation mixture may depend on the polymerase being used. If the interrogated base is complementary to the labeled nucleotide, multiple binding and release events may then be recorded and sufficient statistics be generated to enable determination of the base. In some cases, a change in kinetics relative to that of a normal base may be used to identify if the interrogated base is a modified base.

The polymerases can have certain kinetics such that when Mg²⁺ is added, they can release the nucleotide rather than incorporate it. In some cases, the polymerase may be modified to change the kinetics such that a bound nucleotide is more likely to be incorporated than released when Mg²⁺ is added.

### (C) EXAMPLES

Nucleic acid sequencing with a particle may be accomplished by the following procedure: (1) flush the system with a calcium buffer; (2) bring in the redox solution with the Ca²⁺ buffer and measure the initial level or redox cycling; (3) bring in a solution containing the redox solution and particles with one nucleotide (for example adenine). If the nucleotide is complementary to the interrogated base (thymine), it can bind to the polymerase and be held. If the nucleotide is not complementary to the interrogated base (guanine, cytosine, or adenine), it may not be held by the polymerase; (4) optionally wash out any unbound nucleotides with a redox solution; (5) measure the redox cycling current and use the change from step (2) to determine if the base is a match; (6) bring in Mg²⁺ without any redox solution to allow incorporation of the bound nucleotide; (7) repeat steps (1)-(6) with the next labeled nucleotide; (8) continue until the full sequence is determined or the error rate becomes excessive.

In some embodiments, it may be desirable to utilize long reads or multiple reads with separations between the reads, for example, when sequencing repetitive sequence regions which may otherwise create ambiguous sequence assemblies.

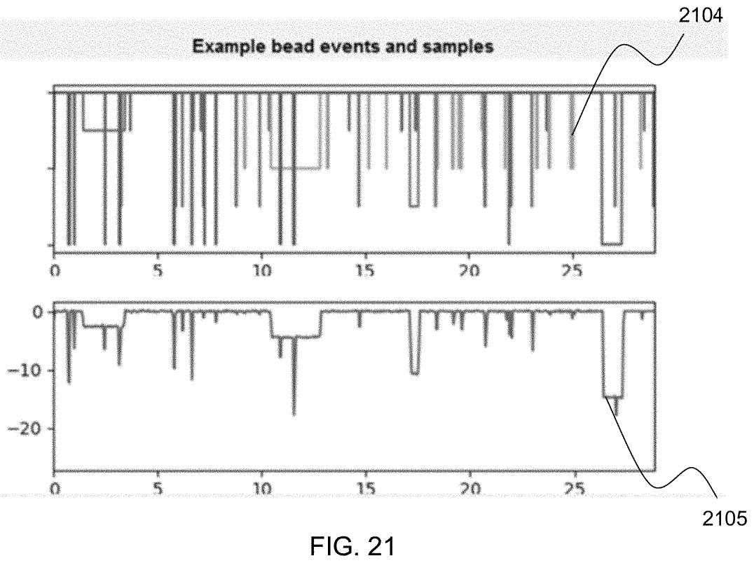

In some cases, an asynchronous chemistry or free running method can be used to take multiple measurements during an indeterminate cycle time. In one example of asynchronous chemistry, four different labels can be used along with divalent cations such as Mg²⁺ or Mn²⁺ that support incorporation of the nucleotide. Since the labeled beads are present during measurement, the signal may include non-binding diffusion events (short) and longer binding and incorporation events. FIG. 21 shows a computer simulation of such a signal. Some of the parameters were derived from experimental data and some were estimated. In the upper panel, the random arrival and duration is shown. In this view, the amplitude of the downward pulse 2104 indicates which label is chosen with larger drops associated with bigger blocking percentages. The lower panel shows the combined effect on the baseline current from all of the blocking events 2105 with the longer drops are associated with labels with a larger blocking efficiency. In experiments, the presence of a bead can inhibit the arrival of a second, especially if the bead is large relative to the sensor well. In this simulation, the arrival of freely diffusing beads does not change the arrival rate so a higher level of free diffusion events is simulated. In this simulation, the software is assumed to identify the longer binding and incorporation event and allow the determination of the base type. With this assumption, the simulator was able to identify the correct base with high accuracy as shown in the confusion matrix shown in FIG. 22. The simulation shows that it is possible to identify bases accurately using an asynchronous or free running method even with free diffusion bead events.

In some cases, a synchronous chemistry method may be employed to take as few as a single measurement during a measure step.

A synchronous system may use one or more different labels. For nucleic acid sequencing 4 different labels can be used. A possible protocol may be as follows:
1. Deliver the redox solution.
2. Measure the redox signal for at least one (e.g., each) sensor.
3. Bring in redox solution with labeled beads (4-label mixture) with Ca²⁺ and wait.
4. Wash the unbound beads out with redox solution with Ca²⁺.
5. Measure the redox signal for at least one (e.g., each) sensor.
6. Bring in Mg²⁺ to incorporate the bound nucleotide, releasing the bead.
7. Repeat steps 1-6.

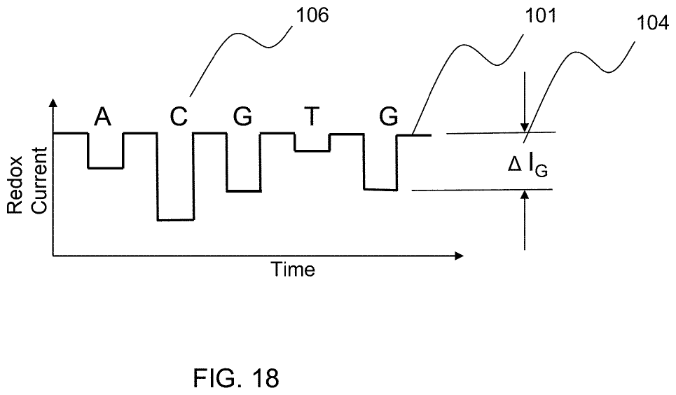

FIG. 18 shows a schematic illustration of a 4-label synchronous current output for a target sequence: TGCAC. The 4-label synchronous baseline readings 101 may be done without any labeled beads present in solution. With Ca²⁺ present, the label may be held for a long time by the polymerase. The complementary nucleotide labeled bead 106 may be held by the polymerase creating a current drop from the baseline which may be measured. For example, the current drop of the G base 104, can be used to identify the complementary nucleotide as a G. The freely diffusing labels may be removed by washing before delivery of divalent cations such as Mg²⁺ or Mn²⁺ that support incorporation of the nucleotide. This can reduce the chance that multiple bases are added during the incorporation step.

A synchronous system may also use two different labels. A possible 2-label protocol may be as follows:
a. Deliver the redox solution.
b. Measure the redox signal for at least one (e.g., each) sensor (B).
c. Bring in 1st set (one nucleotide attached to smaller beads and the other to larger beads) of labeled beads (A, C nucleotides attached) with Ca²⁺ and wait.
d. Wash unbound beads out with a redox solution with Ca²⁺.
e. Measure the redox signal for at least one (e.g., each) sensor.
f. Bring in Mg²⁺ and incorporate any bound nucleotide which releases the bead.
g. Bring in the 2nd set (one nucleotide attached to smaller beads and the other to larger beads) of labeled beads (T, G nucleotides attached) with Ca²⁺ and wait.
h. Wash unbound beads out with a redox solution with Ca²⁺.
i. Measure the redox signal for at least one (e.g., each) sensor.
j. Bring in Mg²⁺ and incorporate any bound nucleotide.
k. Repeat steps a-j.

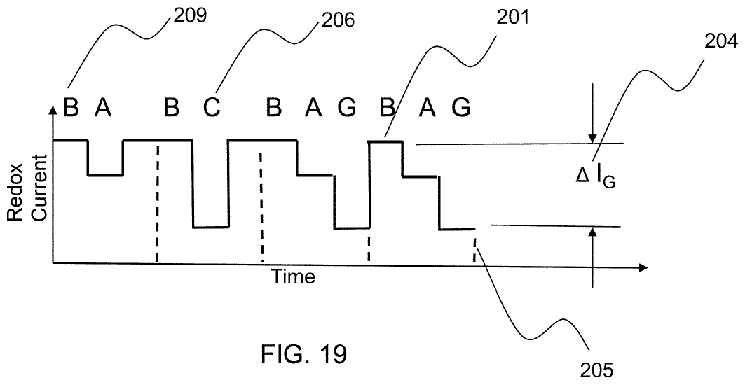

FIG. 19 shows a simulated output for 2-label sequencing for the target sequence TGTCTG. A 2-label system may allow more distinct labels, for example, by having a greater size separation. The 4-label synchronous baseline readings 201 may be made without any labeled beads present in solution. In the simulated output, the baseline reading 209 is signified by a B. With Ca²⁺ present the label may be held for a long time by the polymerase. The complementary nucleotide labeled bead 206 may be held by the polymerase creating a current drop from the baseline which may be measured. For example, the current drop during the second label delivery 204, can be used to identify the complementary nucleotide as a G. The freely diffusing labels may be removed by washing before delivery of divalent cations such as Mg²⁺ or Mn²⁺ that support incorporation of the nucleotide. This can reduce the chance that multiple bases are added during the incorporation step. In the simulated output, for clarity, the completion of a cycle (a-j in protocol) is designated by the vertical dashed lines 205.

A 2-label synchronous system may be made faster by elimination of most of the blank reads. A faster 2-label protocol may be as follows:
a. Bring in 1st set (one nucleotide attached to smaller beads and the other to larger beads) of labeled beads (A, C nucleotides attached) with Ca²⁺ and wait.
b. Wash unbound beads out with a redox solution with Ca²⁺.
c. Measure the redox signal for at least one (e.g., each) sensor.
d. Bring in Mg²⁺ and incorporate any bound nucleotide which releases the bead.
e. Bring in the 2nd set (one nucleotide attached to smaller beads and the other to larger beads) of labeled beads (T, G nucleotides attached) with Ca²⁺ and wait.
f. Wash any unbound beads out with a redox solution with Ca²⁺.
g. Measure the redox signal for at least one (e.g., each) sensor.
h. Bring in Mg²⁺ and incorporate any bound nucleotide.
i. Repeat steps a-h.

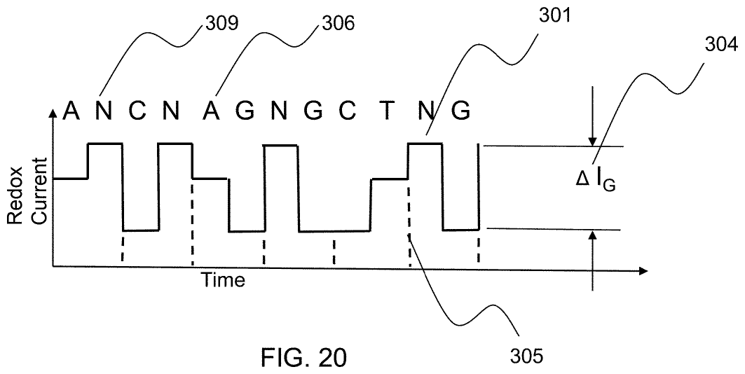

FIG. 20 shows a simulated output for a faster 2-label sequencing for the target sequence TGTCCGAC. In some cases, occasional reads where neither of the two labels have a complementary nucleotide 309 which is represented by a N in the figure can be used to track the baseline 301, reducing the cycle time. With Ca²⁺ present the label may be held for a long time by the polymerase. The complementary nucleotide labeled bead 306 may be held by the polymerase creating a current drop from the baseline which may be measured. For example, the current drop during the second label delivery 304, can be used to identify the complementary nucleotide as a G. The freely diffusing labels may be removed by washing before delivery of divalent cations such as Mg²⁺ or Mn²⁺ that support incorporation of the nucleotide. This can reduce the chance that multiple bases are added during the incorporation step. In the simulated output, for clarity, the completion of a cycle (a-h in protocol) is designated by the vertical dashed lines 305.

In some cases, a skip read method can be used during which a synchronous chemistry method may be utilized for a number of cycles and then suspended, an asynchronous chemistry method may thence be utilized for a period of time and then suspended, followed by a synchronous chemistry method for a number of cycles. Skip reads can allow the generation of multiple reads separated by a longer section of unknown bases. This may facilitate improved sequence assembly similar to that enabled by paired end sequencing. The time during which the asynchronous chemistry method is utilized may be considered to be a “skip period”. Measurements may not be made during a period of time utilizing asynchronous chemistry.

In some cases, an array of sensors may allow a skip period of a skip read method to progress at different rates in different portions of a chip or chips. A difference in rate may result from differences in available concentrations of incorporable nucleotides, differences of concentrations of unincorporable nucleotides, temperature, types or variants of the polymerase, or any other methods to effect the kinetics of incorporation. In some cases, the unincorporable nucleotides can be a nucleotide with substitutions in the phosphate chain such as replacing the alpha, beta or both phosphates with an arsenic, Sn, Bi or N, a PNA nucleotide, an L-DNA nucleotide, a locked DNA nucleotide, a ribonucleotide, an adenine monophosphate, an adenine diphosphate, an adenosine, a deoxyadenosine, a guanine monophosphate, a guanine diphosphate guanosine, a deoxyguanosine, a thymine monophosphate, a thymine diphosphate 5-Methyluridine, a thymidine, a cytosine monophosphate, a cytosine diphosphate cytidine, a deoxycytidine, a uracil monophosphate, a uracil diphosphate, a uridine, or a deoxyuridine.

In cases where a long read is desired, an assay may provide one or more different types of nucleotides while provided with catalytic cations such as magnesium and or manganese for a period of time. Data may or may not be collected. A sequence may or may not be determined, or may be partially determined. During a period of time, an incorporation rate may be much higher than the rates possible with other methods, whereby cycles of noncatalytic periods, catalytic periods, and wash periods may be utilized in alternation. After measurement the current output, the labeled nucleotide may be incorporated. In some cases, a method of the present disclosure may include: bringing in a Ca²⁺ buffer with the labeled nucleotide to hold the labeled nucleotide with the polymerase. Alternatively, the Ca²⁺ buffer can be applied without any labeled nucleotides, so that unbound labeled nucleotides can be removed. This can avoid the problem of multiple nucleotides being added, for example in a homopolymer sequence.

A Mg²⁺ buffer can be provided to allow incorporation of the labeled nucleotide and cleaving of the tether, thereby releasing the particle. This process can continue until an appropriate amount, which is application dependent, of sequencing data from the target nucleotide is obtained.

### (D) COMPUTER CONTROL SYSTEMS

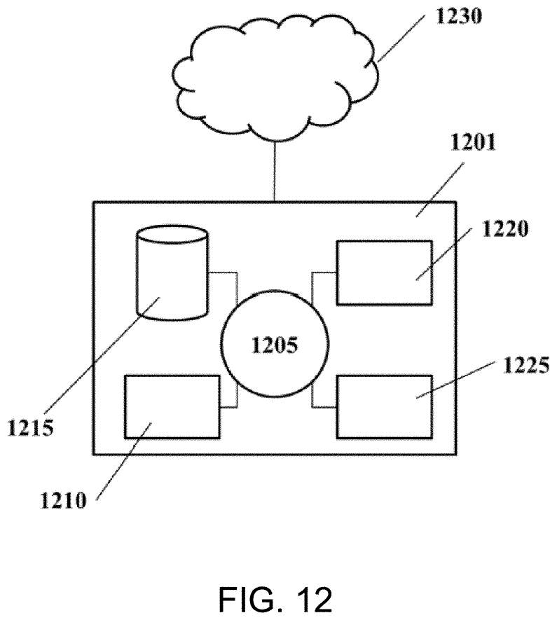

The present disclosure provides computer control systems that are programmed to implement methods of the disclosure. FIG. 12 shows a computer system 1201 that is programmed or otherwise configured to sequence a biomolecule, such as a protein. The computer system 1201 can be the control unit. The computer system 1201 can include a central processing unit (CPU, also “processor” and “computer processor” herein) 1205, which can be a single core or multi core processor, or a plurality of processors for parallel processing. The computer system 1201 can also include memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 In some cases, is a telecommunication and/or data network. The network 1230 can include one or more computer servers, which can enable distributed computing, such as cloud computing. The network 1230, In some cases, with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit can include an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine (or computer) readable medium, such as computer-executable code (or computer program), may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### (E) EMBODIMENTS

Embodiments contemplated herein include embodiments P1 to P145.

Embodiment P1. A method for analyzing a nucleic acid molecule, comprising: (a) providing said nucleic acid molecule coupled to a polymerizing enzyme, wherein said polymerizing enzyme is immobilized adjacent to an electrode of an electrode pair; (b) bringing a reversible redox moiety in contact with said electrode pair, wherein said reversible redox moiety facilitates redox cycling to generate an electrical current between said electrode pair; (c) allowing an interrogating nucleotide, coupled to a particle, to bind to a nucleic acid strand complementary to said nucleic acid molecule with the aid of said polymerizing enzyme, said particle affecting a change in said electric current between said electrode pair, when said interrogating nucleotide is complementary to said nucleic acid molecule; and (d) using said change in said electrical current to identify said interrogating nucleotide, thereby identifying at least a portion of a sequence of said nucleic acid molecule.

Embodiment P2. The method embodiment P1, wherein said electrode pair is part of a plurality of electrode pairs, and wherein at least two electrode pairs of said plurality of electrode pairs comprise a common electrode.

Embodiment P3. The method of embodiment P1 or P2, wherein said electrode pair comprises a nanogap comprising a size between about 100 nanometers (nm) and 200 nm.

Embodiment P4. The method of any one of embodiments P1-P3, wherein said electrode pair is disposed within a cavity, wherein a bottom surface of said cavity comprises a first electrode of said electrode pair, and wherein a rim of said cavity comprises a second electrode of said electrode pair.

Embodiment P5. The method of embodiment P4, wherein a width of said cavity is greater than or equal to a height of said cavity.

Embodiment P6. The method of any one of embodiments P1-P5, wherein said electrical current between said electrode pair is between about 50 picoamperes (pA) and 300 pA.

Embodiment P7. The method of any one of embodiments P1-P6, wherein said electrical current comprises a noise-level of no more than 2 pA.

Embodiment P8. The method of any one of embodiments P1-P7, wherein said particle comprises a dimension that is at least about 70% of a distance between electrodes of said electrode pair.

Embodiment P9. The method of any one of embodiments P1-P8, wherein said polymerizing enzyme comprises a deoxyribonucleic acid (DNA) polymerase, a reverse transcriptase, an RNA-dependent RNA polymerase, an RNA polymerase, or any combination thereof.

Embodiment P10. The method of any one of embodiments P1-P9, wherein said interrogating nucleotide is coupled to said particle by a terminal phosphate of said interrogating nucleotide.

Embodiment P11. The method of any one of embodiments P1-P10, wherein said change in said electric current comprises a decrease in intensity of at least 1% of said electric current.

Embodiment P12. The method of any one of embodiments P1-P11, wherein said change in said electric current comprises a signal-to-noise ratio of at least about 3.

Embodiment P13. The method of any one of embodiments P1-P12, wherein said particle is coupled to at least two types of nucleotides.

Embodiment P14. The method of any one of embodiments P1-P13, further comprising contacting said polymerizing enzyme with a divalent cation to facilitate decoupling of said nucleotide from said particle, thereby releasing said particle.

Embodiment P15. A method for analyzing a nucleic acid molecule, comprising: (a) introducing a solution comprising a reversible redox moiety to an electrode pair having immobilized adjacent thereto a polymerizing enzyme coupled to said nucleic acid molecule, wherein said reversible redox moiety facilitates redox cycling to generate an electrical current between said electrode pair; (b) bringing said nucleic acid molecule in contact with a nucleotide having a particle coupled thereto, under conditions such that said nucleotide couples to a nucleic acid strand complementary to said nucleic acid molecule with aid of said polymerizing enzyme, wherein said particle affects a change in said electrical current; and (c) using said change in said electrical current to identify said nucleotide coupled to said nucleic acid strand, thereby identifying at least a portion of a sequence of said nucleic acid molecule.

Embodiment P16. The method of embodiment P15, wherein at least a portion of said solution is within a well, and wherein at least one electrode of said electrode pair is disposed in said well.

Embodiment P17. The method of embodiment P16, wherein said well is disposed in a substrate comprising a plurality of wells, wherein said plurality of wells comprises a plurality of electrode pairs comprising said electrode pair.

Embodiment P18. The method of embodiment P16 or P17, wherein said nucleic acid molecule is immobilized to an internal surface of said well or to a support in said well.

Embodiment P19. The method of embodiment P16 or P17, wherein said nucleic acid molecule is immobilized to a support of said well.

Embodiment P20. The method of any one of embodiments P16-P19, wherein said polymerizing enzyme is immobilized in the vicinity of or in said well.

Embodiment P21. The method of any one of embodiments P15-P20, wherein said polymerizing enzyme comprises a deoxyribonucleic acid (DNA) polymerase, a reverse transcriptase, an RNA-dependent RNA polymerase, an RNA polymerase, or any combination thereof.

Embodiment P22. The method of any one of embodiments P15-P21, wherein said particle comprises an oxide.

Embodiment P23. The method of any one of embodiments P15-P22, wherein said particle comprises a polymeric material.

Embodiment P24. The method of embodiment P23, wherein said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), and a derivative thereof.

Embodiment P25. The method of any one of embodiments P15-P24, wherein said particle comprises a metallic material.

Embodiment P26. The method of any one of embodiments P15-P25, further comprising bringing said nucleic acid molecule into contact with an additional nucleotide having an additional particle coupled thereto, under conditions such that said additional nucleotide binds to said nucleic acid strand.

Embodiment P27. The method of embodiment P26, wherein said nucleotide and said additional nucleotide are of different base types, and wherein said particle and said additional particle have substantially identical sizes.

Embodiment P28. The method of embodiment P26, wherein said nucleotide and said additional nucleotide are of different base types, and wherein said particle and said additional particle have different sizes.

Embodiment P29. The method of embodiment P28, wherein said nucleotide and said additional nucleotide are of different base types, and wherein said particle and said additional particle have different surface areas.

Embodiment P30. The method of any one of embodiments P15-P29, wherein said nucleotide is coupled to said particle by a tether.

Embodiment P31. The method of embodiment P30, wherein said tether is coupled to said nucleotide by a terminal phosphate of a triphosphate group of said nucleotide such that said particle is decoupled from said nucleotide when said nucleotide is incorporated into said nucleic acid strand.

Embodiment P32. The method of embodiment P30 or P31, wherein said tether comprises a polymeric material.

Embodiment P33. The method of embodiment P32, wherein said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof.

Embodiment P34. The method of any one of embodiments P15-P33, further comprising bringing said nucleic acid molecule into contact with an additional nucleotide having an additional particle coupled thereto, under conditions such that said additional nucleotide binds to said nucleic acid strand, wherein said additional nucleotide is coupled to said additional particle by an additional tether.

Embodiment P35. The method of embodiment P34, wherein said tether and said additional tether have different lengths.

Embodiment P36. The method of any one of embodiments P15-P35, wherein said particle comprises a plurality of polymers on a surface of said particle at a density that reduces a non-specific binding to said surface.

Embodiment P37. The method of embodiment P36, wherein said plurality of polymers comprises a polymer is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof.

Embodiment P38. The method of any one of embodiments P15-P37, further comprising bringing said nucleic acid molecule into contact with an additional nucleotide having an additional particle coupled thereto, under conditions such that said additional nucleotide binds to said nucleic acid strand, wherein said particle and said additional particle comprise said plurality of polymers on surfaces at different densities.

Embodiment P39. The method of any one of embodiments P15-P38, wherein said particle has a charge that electrostatically attracts said reversible redox moiety towards said particle.

Embodiment P40. The method of any one of embodiments P15-P39, further comprising, subsequent to (c), using a lack of a change in said electrical current detected in (b) to identify an abasic site on said nucleic acid strand.

Embodiment P41. The method of any one of embodiments P15-P40, wherein said polymerizing enzyme is contacted with a first buffer comprising a first cation to facilitate a transient binding of said nucleotide with said nucleic acid molecule; and wherein said polymerizing enzyme is subsequently contacted with a second buffer comprising a second cation to facilitate an incorporation of said nucleotide into said nucleic acid strand.

Embodiment P42. The method of embodiment P41, wherein said first cation comprises Ca²⁺, Zn²⁺, Fe²⁺, Be²⁺, Ba²⁺, Cd²⁺, Co²⁺, Cr²⁺, Ni²⁺, Cu²⁺, Pb²⁺, Sr²⁺, or any combination thereof.

Embodiment P43. The method of either of embodiments P41 or P42 wherein said second cation comprises Mg²⁺, Mn²⁺, Zn²⁺, Fe²⁺, Be²⁺, Ba²⁺, Co²⁺, Ni²⁺, Pb²⁺, Sr²⁺, or any combination thereof.

Embodiment P44. The method of embodiment P41, wherein said first cation is Ca²⁺ and said second cation is Mg²⁺.

Embodiment P45. The method of any one of embodiments P41-P44, wherein said change in said electrical current is detected during said transient binding of said nucleotide with said nucleic acid molecule.

Embodiment P46. The method of any one of embodiments P15-P45, wherein said change is an increase or a decrease in said electrical current.

Embodiment P47. The method of any one of embodiments P15-P46, wherein said electrode pair comprises a nanoelectrode.

Embodiment P48. A system for analyzing a nucleic acid molecule, comprising: an electrode pair configured to receive and immobilize adjacent thereto a polymerizing enzyme coupled to said nucleic acid molecule, said electrode pair comprising electrodes separated by a gap, which gap is configured to receive at least part of a solution comprising a reversible redox moiety configured to facilitate redox cycling to generate an electrical current between said nanoelectrodes; a controller operatively coupled to said electrode pair, which said controller is configured to (i) detect a change in said electrical current upon a nucleotide having a particle coupled thereto coming into contact with said nucleic acid molecule, such that said nucleotide binds to a nucleic acid strand complementary to said nucleic acid molecule with the aid of said polymerizing enzyme, wherein said particle effects said change in said electrical current, and (ii) use said change in said electrical current detected in (i) to identify said nucleotide, thereby identifying at least a portion of a sequence of said nucleic acid molecule.

Embodiment P49. The system of embodiment P48, further comprising a well configured to contain at least a part of said solution, wherein said well comprises an electrode of said plurality of electrodes.

Embodiment P50. The system of any one of embodiments P48-P49, wherein said controller is configured to select which electrode pairs are addressed.

Embodiment P51. The system of embodiment any one of embodiments P48-P50, further comprising a fluid flow unit configured to dispense said nucleotide having said particle coupled thereto.

Embodiment P52. The system of any one of embodiments P48-P51, wherein said nucleic acid molecule is immobilized to a surface internal to said well or to a support in said well.

Embodiment P53. The system of embodiment P52, wherein said nucleic acid molecule is immobilized to a of said well.

Embodiment P54. The system of any one of embodiments P49-P53, wherein said polymerizing enzyme is immobilized in the vicinity of or in said well.

Embodiment P55. The system of any one of embodiments P48-P54, wherein said polymerizing enzyme comprises a deoxyribonucleic acid (DNA) polymerase, a reverse transcriptase, an RNA-dependent RNA polymerase, an RNA polymerase, or any combination thereof.

Embodiment P56. The system of any one of embodiments P48-P55, wherein said particle is a solid particle which comprises silicon dioxide.

Embodiment P57. The system of embodiment any one of embodiments P48-P56, wherein said particle comprises a polymeric material.

Embodiment P58. The system of embodiment P57, wherein said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), and a derivative thereof.

Embodiment P59. The system of any one of embodiments P48-P58, wherein said particle comprises a metallic material.

Embodiment P60. The system of any one of embodiments P49-P59, wherein said well is configured to contain an additional nucleotide having an additional particle coupled thereto, under conditions such that said additional nucleotide is incorporated into said nucleic acid strand.

Embodiment P61. The system of embodiment P60, wherein said nucleotide and said additional nucleotide are of different base types, and wherein said particle and said additional particle have substantially identical sizes.

Embodiment P62. The system of embodiment P60, wherein said nucleotide and said additional nucleotide are of different base types, and wherein said particle and said additional particle have different sizes.

Embodiment P63. The system of embodiment P60 or P61, wherein said nucleotide and said additional nucleotide are of different base types, and wherein said particle and said additional particle have different surface areas.

Embodiment P64. The system of any one of embodiments P48-P63, wherein said nucleotide is coupled to said particle by a tether.

Embodiment P65. The system of embodiment P64, wherein said tether is coupled to said nucleotide by a phosphate group of said nucleotide such that said particle is decoupled from said nucleotide when said nucleotide is incorporated into said nucleic acid strand.

Embodiment P66. The system of either of embodiments P64 or P65, wherein said tether comprises a polymeric material.

Embodiment P67. The system of embodiment P66, wherein said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof.

Embodiment P68. The system of any one of embodiments P49-P67, wherein said well is configured to contain an additional nucleotide having an additional particle coupled thereto, under conditions such that said additional nucleotide binds to said nucleic acid strand, wherein said additional nucleotide is coupled to said additional particle by an additional tether.

Embodiment P69. The system of embodiment P68, wherein said tether and said additional tether have different lengths.

Embodiment P70. The system of any one of embodiments P48-P68, wherein said particle comprises a plurality of polymers on a surface of said particle at a density configured to reduce non-specific binding to said surface.

Embodiment P71. The system of embodiment P70, wherein said plurality of polymers comprises a polymer selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof.

Embodiment P72. The system of any one of embodiments P49-P71, wherein said well is configured to contain an additional nucleotide having an additional particle coupled thereto, under conditions such that said additional nucleotide binds to said nucleic acid strand, wherein said particle and said additional particle comprise said plurality of polymers on surfaces at different densities.

Embodiment P73. The system of any one of embodiments P48-P72, wherein said particle has a charge that electrostatically attracts said reversible redox moiety.

Embodiment P74. The system of any one of embodiments P49-P73, wherein said well is configured to contain (iii) a first buffer comprising a first cation in contact with said polymerizing enzyme to facilitate said polymerizing enzyme to transiently bind said nucleotide to said nucleic acid molecule; and (iv) a second buffer comprising a second cation in contact with said polymerizing enzyme to facilitate said polymerizing enzyme to incorporate said nucleotide into said nucleic acid molecule.

Embodiment P75. The system of embodiment P74, wherein said first cation is selected from the group consisting of Ca²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺.

Embodiment P76. The system of either of embodiments P74 or P75, wherein said second cation is selected from the group consisting of Mg²⁺, Mn²⁺, Zn²⁺, Fe²⁺, Be²⁺, Ba²⁺, Cd²⁺, Co²⁺, Cr²⁺, Ni²⁺, Cu²⁺, Pb²⁺ and Sr²⁺.

Embodiment P77. The system of any one of embodiments P48-P76, wherein said change in said electrical current is detected during a transient binding of said nucleotide with said nucleic acid molecule.

Embodiment P78. The system of any one of embodiments P48-P77, wherein said electrode pair comprises a nanoelectrode.

Embodiment P79. A method for analyzing a nucleic acid molecule, comprising: (a) providing a plurality of electrodes and a solution comprising said nucleic acid molecule having a polymerizing enzyme coupled thereto, wherein said plurality of electrodes detects an electrical current through said solution; (b) bringing said nucleic acid molecule into contact with a nucleotide having a particle coupled thereto, under conditions such that said polymerizing enzyme facilitates a transient binding of said nucleotide to a nucleic acid strand complementary to said nucleic acid molecule, wherein said transient binding of said nucleotide having said particle coupled thereto effects a change in said electrical current detected by said plurality of electrodes; (c) subsequent to said transient binding, incorporating said nucleotide into said nucleic acid strand; and (d) using said change in said electrical current detected by said plurality of electrodes to identify said nucleotide transiently bound to said nucleic acid strand, thereby identifying at least a portion of a sequence of said nucleic acid molecule.

Embodiment P80. The method of embodiment P79, wherein said polymerizing enzyme is contacted with a first buffer comprising a first cation to facilitate said transient binding of said nucleotide to said nucleic acid molecule; and wherein said polymerizing enzyme is subsequently contacted with a second buffer comprising a second cation to facilitate incorporation of said nucleotide into said nucleic acid strand.

Embodiment P81. The method of embodiment P80, wherein said first cation is selected from the group consisting of Ca²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺.

Embodiment P82. The method of either of embodiments P80 or P81, wherein said second cation is selected from the group consisting of Mg²⁺, Mn²⁺, Zn²⁺, Fe²⁺, Be²⁺, Ba²⁺, Cd²⁺, Co²⁺, Ni²⁺, Cu²⁺, Pb²⁺ and Sr²⁺.

Embodiment P83. The method of embodiment P80, wherein said first cation is Ca²⁺ and said second cation is Mg²⁺.

Embodiment P84. The method of any one of embodiments P79-P83, wherein said polymerizing enzyme is a deoxyribonucleic acid (DNA) polymerase, a reverse transcriptase, an RNA-dependent RNA polymerase, or an RNA polymerase.

Embodiment P85. The method of embodiment P84, wherein said DNA polymerase is selected from the group consisting of Φ15, Φ29, BS32, B103, Cp-1, Cp-5, Cp-7, GA.-1, G1, L17, M2Y, Nf, PRD1, PZE, PR4, PR5, PR722, PZA, SF5, T4, T7 DNA polymerases, or a functional variant thereof.

Embodiment P86. The method of any one of embodiments P79-P85, wherein said polymerizing enzyme is a modified recombinant polymerase.

Embodiment P87. The method of embodiment P86, wherein said polymerizing enzyme is modified such that it lacks 5′ to 3′ and/or 3′ to 5′ exonuclease activity.

Embodiment P88. The method of any one of embodiments P79-P87, further comprising determining a binding kinetic of said nucleotide to said nucleic acid molecule from said change in said electrical current, and identifying said nucleotide based on said binding kinetics.

Embodiment P89. The method of any one of embodiments P79-P89, wherein said plurality of electrodes comprises a plurality of nanoelectrodes.

Embodiment P90. The method of embodiment P89, wherein said solution is contained in a well, and wherein said well comprises a nanoelectrode of said plurality of nanoelectrodes.

Embodiment P91. A system for analyzing a nucleic acid molecule, comprising: a plurality of electrodes, wherein at least two of said plurality of electrodes are separated from each other by a gap, which gap is configured to receive at least part of a solution comprising said nucleic acid molecule having a polymerizing enzyme coupled thereto; a controller operatively coupled to said plurality of electrodes, which said controller is configured to (i) use said plurality of electrodes to detect a change in said electrical current upon a nucleotide having a particle coupled thereto coming into contact with said nucleic acid molecule, under conditions such that said polymerizing enzyme facilitates a transient binding of said nucleotide to a nucleic acid strand complementary to said nucleic acid molecule, wherein said transient binding of said nucleotide having said particle coupled thereto effects said change in said electrical current, and wherein said nucleotide is incorporated into said nucleic acid strand subsequent to said transient binding; and (ii) use said change in said electrical current detected in (i) to identify said nucleotide, thereby identifying at least a portion of a sequence of said nucleic acid molecule.

Embodiment P92. The system of embodiment P91, wherein said polymerizing enzyme is contacted with a first buffer comprising a first cation to facilitate said transient binding of said nucleotide to said nucleic acid molecule; and wherein said polymerizing enzyme is subsequently contacted with a second buffer comprising a second cation to facilitate an incorporation of said nucleotide into said nucleic acid strand.

Embodiment P93. The system of embodiment P92, wherein said first cation is selected from the group consisting of Ca²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺.

Embodiment P94. The system of embodiment P92 or P93, wherein said second cation is selected from the group consisting of Mg²⁺, Mn²⁺, Zn²⁺, Fe²⁺, Be²⁺, Ba²⁺, Cd²⁺, Co²⁺, Cr²⁺, Ni²⁺, Cu²⁺, Pb²⁺, and Sr²⁺.

Embodiment P95. The system of embodiment P92, wherein said first cation is Ca²⁺ and said second cation is Mg²⁺.

Embodiment P96. The system of any one of embodiments P91-P95, wherein said polymerizing enzyme comprises a deoxyribonucleic acid (DNA) polymerase, a reverse transcriptase, an RNA-dependent RNA polymerase, an RNA polymerase, or any combination thereof.

Embodiment P97. The system of embodiment P96, wherein said DNA polymerase is selected from the group consisting of Φ15, Φ29, BS32, B103, Cp-1, Cp-5, Cp-7, GA-1, G1, L17, M2Y, Nf, PRD1, PZE, PR4, PR5, PR722, PZA, SF5, T4, T7 DNA polymerases, or a functional variant thereof.

Embodiment P98. The system of any one of embodiments P91-P97, wherein said polymerizing enzyme is a modified recombinant polymerase.

Embodiment P99. The system of embodiment P98, wherein said polymerizing enzyme is modified such that it lacks 5′ to 3′ and/or 3′ to 5′ exonuclease activity.

Embodiment P100. The system of any one of embodiments P91-P99, wherein said nucleotide comprises inosine, 5-methylcytosine, pseudouridine, N6-methyladenosine, N1-methyladenosine, 5′ cap-related 7-methylguanosine, and/or 2′-O-methylation.

Embodiment P101. The system of embodiment any one of embodiments P91-P100, wherein binding kinetics of said nucleotide to said nucleic acid molecule is determined from said change in said electrical current, and wherein said binding kinetics is used to identify said nucleotide.

Embodiment P102. The system of embodiment P101, wherein said binding kinetics are used to determine a modification state of a nucleotide of said nucleic acid molecule.

Embodiment P103. The method of any one of embodiments P91-P102, wherein said solution is contained in a well, and wherein said well comprises a nanoelectrode of said plurality of electrodes.

Embodiment P104. A method for detecting a biomolecule, comprising: (a) providing a plurality of electrodes and a solution comprising a reversible redox moiety that is configured to facilitate redox cycling and to generate an electrical current between electrodes of said plurality of electrodes, wherein an individual electrode of said plurality of electrodes comprises a surface having a molecule comprising an affinity for said biomolecule coupled thereto; (b) directing said biomolecule to said molecule; (c) detecting a change in said electrical current using said plurality of electrodes upon said biomolecule coming into contact with said molecule; and (d) using said change in said electrical current detected in (c) to identify said biomolecule.

Embodiment P105. The method of embodiment P104, wherein (c) comprises detecting said change in said electrical current using said plurality of electrodes upon binding of said biomolecule to said molecule

Embodiment P106. The method of either of embodiments P104-P105, wherein at least a portion of said solution is contained in a well, and wherein said well comprises said electrode of said plurality of electrodes.

Embodiment P107. The method of any one of embodiments P104-P106, wherein said plurality of electrodes comprises an additional electrode external to said well.

Embodiment P108. The method of any one of embodiments P104-P107, wherein said plurality of electrodes comprises a plurality of nanoelectrodes.

Embodiment P109. The method of any one of embodiments P104-P108, further comprising using a fluid flow unit configured to dispense said biomolecule.

Embodiment P110. The method of any one of embodiments P104-P109, wherein said biomolecule comprises a protein or a polypeptide.

Embodiment P111. The method of any one of embodiments P104-P109, wherein said biomolecule comprises a nucleic acid molecule.

Embodiment P112. The method of any one of embodiments P104-P111, wherein an additional electrode of said plurality of electrodes comprises an additional molecule, and wherein said change in said electrical current is detected upon an additional biomolecule coming into contact with said additional molecule.

Embodiment P113. The method of embodiment P112, wherein said biomolecule comprises a particle coupled thereto, and wherein said additional biomolecule comprises an additional particle coupled thereto.

Embodiment P114. The method of embodiment P113, wherein said biomolecule and said additional biomolecule are different, and wherein said particle and said additional particle have substantially identical sizes.

Embodiment P115. The method of embodiment P113, wherein said biomolecule and said additional biomolecule are different, and wherein said particle and said additional particle have different sizes.

Embodiment P116. The method of either of embodiments P113 or P115, wherein said biomolecule and said additional biomolecule are different, and wherein said particle and said additional particle have different surface areas.

Embodiment P117. The method of any one of embodiments P113-P116, wherein said biomolecule and said additional biomolecule are different, and wherein said biomolecule and said additional biomolecule are coupled to said particle by tethers.

Embodiment P118. The method of embodiment P117, wherein said tethers comprise a polymeric material.

Embodiment P119. The method of embodiment P118, wherein said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof.

Embodiment P120. The method of any one of embodiments P117-P119, wherein said tethers have different lengths.

Embodiment P121. The method of any one of embodiments P104-P120, wherein said particle comprises a plurality of polymers on a surface of said particle at a density such that said plurality of polymers reduces non-specific binding to said surface.

Embodiment P122. The method of embodiment P121, wherein said plurality of polymers comprises a polymer selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof.

Embodiment P123. The method of any one of embodiments P113-P122, wherein said particle and said additional particle comprise a plurality of polymers on surfaces at different densities.

Embodiment P124. The method of any one of embodiments P104-P123, wherein said particle has a charge that electrostatically attracts said reversible redox moiety.

Embodiment P125. A system for biomolecule detection, comprising: a plurality of electrodes, wherein an individual electrode of said plurality of electrodes comprises a surface having a molecule specific for said biomolecule coupled thereto, wherein at least two of said plurality of electrodes are separated from each other by a gap, which gap is configured to receive at least part of a solution comprising a reversible redox moiety that is configured to facilitate redox cycling and to generate an electrical current between said at least two of said plurality of electrodes; and a controller operatively coupled to said plurality of electrodes, which said controller is configured to (i) use said plurality of electrodes to detect a change in said electrical current upon said biomolecule being directed to and coming into contact with said molecule; and (ii) use said change in said electrical current detected in (i) to identify said biomolecule.

Embodiment P126. The system of embodiment P125, wherein said plurality of electrodes comprises a plurality of nanoelectrodes.

Embodiment P127. The system of embodiment P125 or P126, further comprising a well configured to contain at least part of said solution, wherein said well comprises said individual electrode of said plurality of electrodes.

Embodiment P128. The system of embodiment P127, wherein said plurality of electrodes comprises an additional electrode external to said well.

Embodiment P129. The system of any one of embodiments P125-P128, further comprising a fluid flow unit configured to dispense said biomolecule.

Embodiment P130. The system of any one of embodiments P125-P129, wherein said biomolecule comprises a protein or a polypeptide.

Embodiment P131. The system of any one of embodiments P125-P129, wherein said biomolecule comprises a nucleic acid molecule.

Embodiment P132. The system of embodiment P128, wherein said additional electrode comprises an additional molecule coupled to a surface of said additional electrode, wherein said well is configured to contain an additional biomolecule, wherein said change in said electrical current is detected upon said additional biomolecule coming into contact with said additional molecule.

Embodiment P133. The system of embodiment P132, wherein said biomolecule comprises a particle coupled thereto, and wherein said additional biomolecule comprises an additional particle coupled thereto.

Embodiment P134. The system of embodiment P133, wherein said particle and said additional particle effect said change in said electrical current.

Embodiment P135. The system of embodiment P133 or P134, wherein said biomolecule and said additional biomolecule are different, and wherein said particle and said additional particle have substantially identical sizes.

Embodiment P136. The system of embodiment P133 or P134, wherein said biomolecule and said additional biomolecule are different, and wherein said particle and said additional particle have different sizes.

Embodiment P137. The system of any one of embodiments P133, P134, or P136, wherein said biomolecule and said additional biomolecule are different, and wherein said particle and said additional particle have different surface areas.

Embodiment P138. The system of any one of embodiments P133-P137, wherein said biomolecule and said additional biomolecule are different, and wherein said biomolecule and said additional biomolecule are coupled to said particle and said additional particle via tethers.

Embodiment P139. The system of embodiment P138, wherein said tethers comprise a polymeric material.

Embodiment P140. The system of embodiment P139, wherein said polymeric material is selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and a derivative thereof.

Embodiment P141. The system of any one of embodiments P138-P140, wherein said tethers have different lengths.

Embodiment P142. The system of any one of embodiments P125-P141, wherein said particle comprises a plurality of polymers on a surface of said particle at a density configured to reduce non-specific binding to said surface.

Embodiment P143. The system of embodiment P142, wherein said plurality of polymers comprises a polymer selected from the group consisting of polyethylene glycol (PEG), polyethylenimine (PEI), polylactic acid (PLA), polytetrafluoroethylene (PTFE), polyvinylidene fluoride (PDVF), fluorinated ethylene propylene (FEP), perfluoroalkoxy alkane (PFA), polyethylenetetrafluoroethylene (ETFE), polychlorotrifluoroethylene (PCTFE), poly(methyl methacrylate) (PMMA), polydimethylsiloxane (PDMS), cyclic olefin copolymer (COC), polystyrene (PS), polyacrylonitrile (PAN), polyethylene terephthalate (PET), an alkane polymer, and any combination thereof.

Embodiment P144. The system of any one of embodiments P133-P143, wherein said particle and said additional particle comprise a plurality of polymers on surfaces at different densities.

Embodiment P145. The system of any one of embodiments P133-P144, wherein said particle has a charge that electrostatically attracts said reversible redox moiety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for analyzing a nucleic acid molecule, comprising:
(a) providing said nucleic acid molecule coupled to a polymerizing enzyme, wherein said polymerizing enzyme is immobilized adjacent to an electrode of an electrode pair;
(b) bringing a reversible redox moiety in contact with said electrode pair, wherein said reversible redox moiety facilitates redox cycling to generate an electrical current between said electrode pair;
(c) allowing an interrogating nucleotide, coupled to a particle, to bind to a nucleic acid strand complementary to said nucleic acid molecule with the aid of said polymerizing enzyme, said particle effecting a change in said electric current between said electrode pair, when said interrogating nucleotide is complementary to said nucleic acid molecule; and
(d) using said change in said electrical current to identify said interrogating nucleotide, thereby identifying at least a portion of a sequence of said nucleic acid molecule.

2. The method of claim 1, wherein said electrode pair is part of a plurality of electrode pairs, and wherein at least two electrode pairs of said plurality of electrode pairs comprise a common electrode.

3. The method of claim 1, wherein said electrode pair comprises a nanogap comprising a size between about 10 nanometers (nm) and 200 nm.

4. The method of claim 1, wherein said electrode pair is disposed within a cavity, wherein a bottom surface of said cavity comprises a first electrode of said electrode pair, and wherein a rim of said cavity comprises a second electrode of said electrode pair.

5. The method of claim 1, wherein said polymerizing enzyme comprises a deoxyribonucleic acid (DNA) polymerase, a reverse transcriptase, an RNA-dependent RNA polymerase, an RNA polymerase, or any combination thereof.

6. The method of claim 1 wherein said interrogating nucleotide is coupled to said particle by a terminal phosphate of said interrogating nucleotide.

7. The method of claim 1, further comprising contacting said polymerizing enzyme with a divalent cation to facilitate decoupling of said nucleotide from said particle, thereby releasing said particle.

8. A method for analyzing a nucleic acid molecule, comprising:
(a) introducing a solution comprising a reversible redox moiety to an electrode pair having immobilized adjacent thereto a polymerizing enzyme coupled to said nucleic acid molecule, wherein said reversible redox moiety facilitates redox cycling to generate an electrical current between said electrode pair;
(b) bringing said nucleic acid molecule in contact with a nucleotide having a particle coupled thereto, under conditions such that said nucleotide couples to a nucleic acid strand complementary to said nucleic acid molecule with aid of said polymerizing enzyme, wherein said particle effects a change in said electrical current; and
(c) using said change in said electrical current to identify said nucleotide coupled to said nucleic acid strand, thereby identifying at least a portion of a sequence of said nucleic acid molecule.

9. The method of claim 8, wherein at least a portion of said solution is within a well, and wherein at least one electrode of said electrode pair is disposed in said well.

10. The method of claim 8, further comprising bringing said nucleic acid molecule into contact with an additional nucleotide having an additional particle coupled thereto, under conditions such that said additional nucleotide couples to said nucleic acid strand.

11. The method of claim 10, wherein said nucleotide and said additional nucleotide are of different base types, and wherein said particle and said additional particle have substantially identical sizes.

12. The method of claim 10, wherein said nucleotide and said additional nucleotide are of different base types, and wherein said particle and said additional particle have different sizes.

13. The method of claim 8, wherein said nucleotide is coupled to said particle by a tether, and wherein said tether is coupled to said nucleotide by a terminal phosphate of a phosphate group of said nucleotide such that said particle is decoupled from said nucleotide when said nucleotide is incorporated into said nucleic acid strand.

14. The method of claim 8, wherein said polymerizing enzyme is contacted with a first buffer comprising a first cation to facilitate a transient binding of said nucleotide with said nucleic acid molecule; and wherein said polymerizing enzyme is subsequently contacted with a second buffer comprising a second cation to facilitate an incorporation of said nucleotide into said nucleic acid strand.

15. The method of claim 14, wherein said change in said electrical current is detected during said transient binding of said nucleotide with said nucleic acid molecule.

16. A system for analyzing a nucleic acid molecule, comprising:
an electrode pair configured to receive and immobilize adjacent thereto, a polymerizing enzyme coupled to said nucleic acid molecule, said electrode pair comprising electrodes separated by a gap, which gap is configured to receive at least part of a solution comprising a reversible redox moiety configured to facilitate redox cycling to generate an electrical current between said nanoelectrodes;
a controller operatively coupled to said electrode pair, which said controller is configured to (i) detect a change in said electrical current upon a nucleotide having a particle coupled thereto coming into contact with said nucleic acid molecule, such that said nucleotide binds to a nucleic acid strand complementary to said nucleic acid molecule with the aid of said polymerizing enzyme, wherein said particle effects said change in said electrical current, and (ii) use said change in said electrical current detected in (i) to identify said nucleotide, thereby identifying at least a portion of a sequence of said nucleic acid molecule.

17. The system of claim 16, further comprising a well configured to contain at least a part of said solution, wherein said well comprises an electrode of said plurality of electrodes.

18. The system of claim 16, wherein said controller is configured to select which electrode pairs are addressed.

19. A method for detecting a biomolecule, comprising:
(a) providing a plurality of electrodes and a solution comprising a reversible redox moiety that is configured to facilitate redox cycling and to generate an electrical current between electrodes of said plurality of electrodes, wherein an individual electrode of said plurality of electrodes comprises a surface having a molecule which has an affinity for said biomolecule coupled thereto;
(b) directing said biomolecule to said molecule;
(c) detecting a change in said electrical current using said plurality of electrodes upon said biomolecule coming into contact with said molecule; and
(d) using said change in said electrical current detected in (c) to identify said biomolecule.

20. The method of claim 19, wherein (c) comprises detecting said change in said electrical current using said plurality of electrodes upon binding of said biomolecule to said molecule.
